(19) United States
(12) Patent Application Publication
(10) Pub. No.: US 2022/0183843 A1
(43) Pub. Date: Jun. 16, 2022

(54) METHODS AND DEVICES FOR DELIVERING IMPLANTABLE PROSTHESES

(71) Applicant: Elixir Medical Corporation, Milpitas, CA (US)

(72) Inventors: Motasim SIRHAN, Los Altos, CA (US); John YAN, Los Gatos, CA (US); Vinayak BHAT, Cupertino, CA (US); Joseph PARASCHAC, Campbell, CA (US); Benjamyn SERNA, Gilroy, CA (US)

(73) Assignee: Elixir Medical Corporation, Milpitas, CA (US)

(21) Appl. No.: 17/684,874

(22) Filed: Mar. 2, 2022

Related U.S. Application Data
(63) Continuation of application No. PCT/US20/48885, filed on Sep. 1, 2020.
(60) Provisional application No. 62/895,388, filed on Sep. 3, 2019.
(60) Provisional application No. 63/002,580, filed on Mar. 31, 2020.

(51) Int. Cl. A61F 2/24

(52) U.S. Cl. CPC A61F 2/2466

(57) ABSTRACT

A catheter system for delivering an anchor to a valve annulus in a heart valve includes a locating catheter and an anchor delivery catheter. The locating catheter has a distal end which can be advanced beneath the valve annulus near a target site on an upper surface of the valve annulus. The anchor delivery catheter has a distal end which can be advanced over the valve annulus to deliver an anchor along a delivery path to the target site. The distal end of the locating catheter has at least one magnetic element, and the distal end of the anchor delivery catheter has at least one magnetic element which attracts the at least one magnetic element on the locating catheter to pivotally couple the distal end of the anchor delivery catheter to the distal end of the locating catheter. In this way, the deliver catheter can be pivoted relative to the locating catheter to orient the delivery path away from the locating catheter.

### CROSS-REFERENCE

This application is a continuation of PCT Application No. PCT/US2020/048885 (Attorney Docket No. 32016-721.601), filed Sep. 1, 2020, which claims the benefit of Provisional No. 62/895,388 (Attorney Docket No. 32016-718.102), filed Sep. 3, 2019, and of Provisional No. 63/002,580 (Attorney Docket No. 32016-721.101), filed Mar. 31, 2020, the full disclosures of which are incorporated herein by reference.

### BACKGROUND OF THE INVENTION

Field of the Invention. The present invention generally relates generally to medical devices and methods, particularly those in the field of cardiology. More particularly, the present invention relates systems and methods for access heart valves for treatment, repair, or replacement.

Heart valves have important biological function, with a wide range of anatomical configuration including shapes, designs, and dimensions, and are subject to an array of different conditions such as disease conditions that can cause impairment or malfunction. The mitral valve, for example, consists of an annulus containing anterior and posterior leaflets located at the junction between the left atrium and the left ventricle. The valve leaflets are attached to the left ventricle heart papillary muscles via chordae tendineae. Valvular impairment or dysfunction can be caused or exacerbated by changes to the valve configuration including shape, size, and dimension of the valve (or annulus), the length or functionality of the chordae, the leaflets function, causing impairment or dysfunction of the valve.

A variety of cardiac surgical procedures are routinely performed, including for example, surgical annuloplasty, implantation of artificial chordae or repair of chordae, and resection leaflet surgical valve repair. These procedures are performed typically via open heart typically using bypass surgery, including opening the patient's chest and heart, a risky and invasive procedure with long recovery times and associated complications.

As an alternative to such open-heart procedures, less-invasive surgical and percutaneous devices and procedures are being developed to replace or repair the mitral valve. Less invasive surgical and percutaneous options for valve repair typically attempt to replicate more invasive surgical techniques. Many such devices, however, have one or more disadvantages, such as a large size, complex use, limited efficacy, and limited applicability to different anatomical valve configuration.

For these reasons, the results of many percutaneous and less-invasive cardiac procedures, particularly those performed on the mitral valve, have proven to be inferior to open surgical valve repair procedures. Such inferior results often result from limited visualization of the heart valve anatomy during percutaneous and less-invasive cardiac procedures. No single imaging modality provides all anatomical information necessary. Ultrasonic imaging methods do a good job of showing tissue sections, but a poor job of showing the position of the interventional tools in relation to imaged tissue. In contrast, fluoroscopic imaging reveals the tool positions well, but images tissue poorly.

What is needed therefore are devices, tools, systems, and methods for use in or with less-invasive surgical and percutaneous techniques, particularly those performed on a beating heart, and more particularly those performed for mitral valve repair and replacement. Such devices, tools, systems, and methods should preferably address valve regurgitation, minimize or eliminate device migration, be applicable to broader patient population having various valve configurations, while overcoming the limits of current imaging technology. The inventions herein meet at least some of these needs.

2. Listing of the Background Art. Commonly owned PCT/US2019/032976 describes systems and methods for reshaping a valve annulus using an elongate template that is attached to the annulus.

### SUMMARY OF THE INVENTION

The present invention comprises devices and methods for less invasive surgical and/or percutaneous treatment or repair of a body organ, lumen, cavity, or annulus. In a preferred example, the present invention comprises devices and methods for open surgical, less invasive surgical, and percutaneous treatment or repair of heart valves comprising valve annulus and valve leaflets. An example of heart valves comprises aortic, mitral, pulmonary, and tricuspid valves. Although certain examples show a specific valve, the inventions described and claimed herein are applicable to all valves in the body and additionally other body annulus, lumen, cavity, and organs.

In one example, an elongate device has one or more locating elements, such as whiskers, flaps, feelers, wires, sensors, or the like, extending from an engagement end or region near a portion of the device. The locating elements typically extend outwardly and distally relative to a central axis of a shaft or other body of the elongate device. Locating elements may be formed from any materials capable of engaging tissue and locating the catheter body in an advantageous position during a surgical procedure. Suitable polymers include pebax, nylon, abs, ePTFE, or the like, hydrogels, metals including Nitinol, stainless steel, cobalt chrome or the like, or composite materials. They may be constructed with radiopaque additives including barium sulfate, bismuth subcarbonate, bismuth oxychloride, tungsten, or the like. They may have radiopaque markers disposed along their length, including platinum bands, radiopaque inks, or sections of polymers with radiopaque additives. They may be constructed with echogenic features including hollow glass beads, air pockets, or two or more materials of different stiffness or density. They may be constructed with echogenic surface finishes which may include bead blasting, surface texturing, or retro-reflective textures (including hemispheres or corner cube shapes). Dimensions of the locating elements will be dependent on the exact application but will generally have a thickness between 0.1 mm and 1 mm, a width between 0.5 mm and 2 mm, and a length between 1 mm and 20 mm.

In additional examples, the locating elements make take the form of a basket having an adjustable diameter. The basket may be disposed proximal or partially proximal to the tissue anchor, and be configured to interact with the tissue in the vicinity of the valve annulus, typically the wall of a chamber of the heart, typically the wall of the right atrium. The radius of the basket may be adjustable from as little as 5 mm to as much as 20 mm, and may include echogenic features to enhance visibility of the central tissue anchor delivery section of the elongate device. Adjusting the radius of the basket can position the tissue anchor delivery section of the elongate device at a desired distance from the interacting tissue or wall of the chamber of the heart.

In a further example, one or more semi-rigid locating elements can be deployed to assume a shape conducive to interacting with the walls of one or more chambers of the heart to locate the tissue anchor delivery section of the elongate device. Typically, one semi-rigid locating element will establish a fixed distance and/or angle from the locating tissue feature to the tissue anchor delivery section of the elongate device. It is also possible that a second, third, or larger number of semi-rigid locating elements will establish contact points on other anatomical features in the region of the valve annulus, including valve leaflets, the valve annulus, the wall of an adjacent chamber, chordae, papillary muscles, and the like. A combination of two or more locating elements could in this fashion locate the axis of the elongate device at an advantageous angular alignment and location relative to a target tissue, typically a valve annulus.

In one further example, an elongate device configured for delivering a tissue anchor has an attached magnet or magnetic material. An elongate locating target device places a target magnet or magnetic material in a body lumen or cavity in the vicinity of the target tissue. This cavity is typically a chamber of the heart and may include the right or left ventricle, and/or the right or left atrium. The elongate device for delivering a tissue anchor is brought into the field of the target magnet of the locating target device and is thereby stabilized in location for placing a tissue anchor.

In a first aspect, a catheter system in accordance with the principles of the present invention for delivering an anchor to a valve annulus in a heart valve comprises a locating catheter, typically configured to be advanced to and positioned within a heart ventricle, and an anchor delivery catheter, typically configured to be advanced to and positioned within a heart atrium. The locating catheter typically has a distal end configured to be advanced beneath the valve annulus near a target site on the valve annulus, while the anchor delivery catheter typically has a distal end configured to be advanced approximate the valve annulus to deliver an anchor along a delivery path to the target site. The distal end of the locating catheter is configured with at least one magnetic element, and the distal end of the anchor delivery catheter is also configured with at least one magnetic element, where the at least one magnetic element on the locating catheter attracts the at least one magnetic element on the anchor delivery catheter. In particular examples, the distal end of the anchor delivery catheter magnetically couples to the distal end of the locating catheter to form a “virtual fulcrum,” where the deliver catheter can be pivoted relative to the locating catheter to orient the delivery path away from the locating catheter and toward the valve annulus, typically in a laterally outward direction (away from the root of the aorta or the septum) so that the anchor can be implanted in a central portion of the valve annulus spaced outwardly from the base of a leaflet based attached to an inner periphery of the annulus.

In particular examples, the locating catheter of the catheter systems of the present invention may be configured to be placed into a ventricle and the anchor delivery catheter may configured to be placed in the atrium, and more particularly the locating catheter may configured to be placed into a left ventricle beneath a mitral valve annulus (and often an adjacent posterior leaflet) and the anchor delivery catheter is configured to be placed in the left atrium above the mitral valve annulus.

The magnetic elements of the systems of the present invention may be arranged in a variety of ways. Often, each of the magnetic elements comprises a magnet, but in other instances one of the magnetic elements may comprise a magnet and the other of the magnetic elements may comprises a magnetizable structure. In some instances, each magnet may be axially polarized, e.g. with a distal tip of the magnet on the delivery catheter having a polarity opposite to a polarity of the distal tip of the magnet on the locating catheter. In other instances, one of the magnets may be axially polarized and the other magnet is radially polarized. In some examples, at least one of the magnets comprises a shell magnet, e.g. where the shell magnet at least partially surrounds a delivery lumen on the anchor delivery catheter. In some examples, a delivery lumen on the anchor delivery catheter passes at least partially through a lumen the magnet on the anchor delivery catheter. In many instances, the magnet on the locating catheter comprises a blunt tip magnet.

In certain instances, a distal end of the locating catheter may be configured to fit between a valve leaflet and a ventricular wall when the valve is open, allowing the valve function without causing significant stenosis of the heart valve. The locating catheter may comprise a visual alignment marker at its distal tip, where the visual alignment marker is typically configured to allow a user to align the distal end of the locating catheter with the target site while the locating catheter is being advanced under visualization. Such visual alignment markers may comprise an optical, a fluoroscopic, or an echogenic marker. Such visual alignment markers may additionally or alternatively comprise a plurality of laterally extending arms. Such laterally extending arms may be straight, may be curved, may have both straight and curved regions, or may have many other particular geometries. Curved or other arms may be on an axially extendable shaft.

In other instances, the anchors delivered by the anchor delivery catheter may comprise one or more helical anchors detachably secured to a rotatable drive shaft located in a lumen of the anchor delivery catheter.

In a second aspect, methods in accordance with the principles of the present invention for delivering an anchor to a heart valve annulus comprise advancing a distal end of a locating catheter into a heart ventricle to a location beneath the valve leaflet near a target site on of the valve annulus. A distal end of an anchor delivery catheter is advanced to a location over the upper surface of the valve adjacent the target site where the anchor delivery catheter is configured to deliver a helical or other anchor along a delivery path. The distal end of the anchor delivery catheter is magnetically coupled to the distal end of the locating catheter across the mitral valve, and the anchor is advanced along the delivery path to the target site.

In specific instances, the anchor delivery catheter is manipulated to align the delivery path with the target site on the valve annulus, where the magnetic coupling allows the distal end of the anchor delivery catheter to pivot relative to the distal end of the locating catheter while manipulating the anchor delivery catheter. As explained in more detail below, the coupling of the magnetic elements across the valve annulus near the attachment location of the valve leaflet forms a “virtual fulcrum” which enables the desired pivotal manipulation to allow “aiming” of the delivery path to a desired target sire in the valve annulus.

In some instances, the anchor delivery catheter is delivered through an introducer sheath into the left atrium. When using the introducer, manipulating may comprise axially advancing and retracting the anchor delivery catheter through the introducer sheath to pivot the distal end of the anchor delivery catheter and change the direction of the delivery path. Often, manipulating may comprise advancing and retracting a distal end of the introducer sheath within the atrium to pivot the distal end of the anchor delivery catheter and change the direction of the delivery path. In particular instances, (1) the distal end of the locating catheter may be aligned along a wall of the ventricle and (2) the distal end of the anchor delivery catheter and the delivery path may be directed laterally outwardly relative to the locating catheter so that the delivery path intersects the annulus at a target site located radially outwardly of the catheter. In further particular instances, the heart valve annulus may comprise a mitral valve annulus. In other particular instances, the heart valve annulus may comprise an aortic valve annulus, a pulmonary valve annulus, or a tricuspid valve annulus.

Delivery may comprise rotating at least one helical anchor to implant the anchor into the annular tissue. Alternatively, the anchors may comprise barbs, hooks, t-tags, sutures, and other and other devices which may be implanted from the distal end of the anchor delivery catheter in a variety of ways.

Often, the methods for delivering anchors according to the present invention may be performed under visualization. For example, the locating catheter may comprise flexible extensions extending radially from the catheter body approximate the distal tip of the catheter, where the flexible extensions are visualized to assist in positioning the locating catheter beneath the valve leaflet. The flexible extensions may be visualized using fluoroscopy and/or using ultrasonography, where visualizing the extensions on the locating catheter is used to align an implant coupled to the atrial tissue anchor.

Often, the present invention provides a mitral valve annulus anchor delivery catheter system comprising a ventricular locating catheter and an atrial anchor delivery catheter. The ventricular locating catheter has a distal end configured to be advanced beneath the mitral valve annulus near a target site on an upper surface of the mitral valve annulus. The atrial anchor delivery catheter has a distal end configured to be advanced over the mitral valve annulus to deliver an anchor to the target site. The distal end of the ventricular locating catheter and the distal end of the atrial anchor delivery catheter are configured with magnetic elements that attract each other to orient the distal end of the atrial anchor delivery catheter to deliver the anchor in a laterally outward direction and into the mitral valve annulus. As described further with respect to the anchor delivery methods below, such magnetic coupling acts as a pivot or hinge to allow a user to reorient a delivery direction or path of the atrial anchor delivery catheter relative to the stationary ventricular locating catheter. In some instances, the anchor may be delivered from the ventricular locating catheter while the atrial anchor delivery catheter locates the target site.

Typically, at least one of the locating catheter and the anchor delivery catheter will comprise at least one magnetic element, typically near a distal end thereof, while the other of the catheters will comprise at least one magnetic or “magnetizable” element. Magnetizable elements comprise materials attracted to magnets, including ferromagnetic materials, such as iron, cobalt, nickel, and rare earth metals; paramagnetic materials such as neodymium, strontium, and yttrium; and ferrimagnetic compounds such as ferrite; particles of these magnetizable materials embedded in one or more polymers and epoxies; as well as compounds including these materials, such as compounds of iron oxide and strontium carbonate, or of neodymium iron and boron. Magnetic elements will usually comprise permanent magnets, such as neodymium, rare earth, samarium cobalt, alnico, ferrite/ceramic magnets or others known in the art, but also including electromagnets.

The magnetic coupling structure can take a variety of forms. In some instances, the magnetic elements are both magnets. In some instances, one of the magnetic elements may comprise a magnet and the other comprises a magnetizable element. In the case of a magnet on each catheter, both magnets may be axially polarized with opposite poles at each respective distal tip, one of the magnets may be axially polarized and the other may be radially polarized, both magnets may be radially polarized with opposite poles at one surface of each respective distal tip, either or both magnets comprise a shell magnet, and the magnets on the ventricular locating catheter may comprises a blunt tip magnet.

In still other instances, the ventricular locating catheter comprises a visual alignment marker at its distal tip, where the visual alignment marker may comprise an optical, a fluoroscopic, or an echogenic marker. In specific instances, the visual alignment marker may comprise a pair of laterally extending arms, where the arms may be straight, may be curved, or may have other configurations. Curved and other arms may be deployed on a separate shaft, for example an axially extendable shaft disposed in a lumen of the catheter. In a specific implementation, the anchor comprises a helical anchor on a rotatable drive shaft located in a lumen of the atrial anchor delivery catheter.

The present invention provides still further provides a method for delivering an anchor to a mitral valve annulus anchor. A distal end of a ventricular locating catheter is advanced to a location beneath the mitral valve annulus near a target site on an upper surface of the mitral valve annulus. A distal end of an atrial anchor delivery catheter is advanced to a location over the upper surface of the mitral valve annulus adjacent the target site, where the atrial anchor delivery catheter is configured to deliver an anchor along a delivery path. The distal end of the atrial anchor delivery catheter is magnetically coupled to the distal end of the ventricular locating catheter across the mitral valve annulus. The atrial anchor delivery catheter can then be manipulated to aim the delivery path at the target site on the valve annulus, wherein the magnetic coupling allows the distal end of the atrial anchor delivery catheter to pivot relative to the distal end of the ventricular locating catheter. Once the delivery path is properly aligned, the anchor may be along the delivery path to the target site. In particular embodiments, the atrial anchor delivery catheter may be delivered through an introducer sheath into the left atrium. In such instances, manipulating the atrial anchor delivery catheter may comprise axially advancing and retracting the atrial anchor delivery catheter through the delivery sheath to pivot the distal end of the atrial anchor delivery catheter and change the direction of the delivery path. Additionally or alternatively, manipulating the atrial anchor delivery catheter may comprise advancing and retracting a distal end of the delivery sheath within the atrium to pivot the distal end of the atrial anchor delivery catheter and change the direction of the delivery path. In still further, (1) the distal end of the ventricular locating catheter may aligned along a wall of the ventricle and (2) the distal end of the atrial anchor delivery catheter and the delivery path are directed laterally outwardly relative to the cranial-caudal direction so that the delivery path intersects the annulus at a target site located radially outwardly of the catheter.

In another example, a basket formed of a multitude of semi-rigid wires, typically 3 or more, can be located against the valve annulus in a stable configuration. One or more of the wires of the basket can then be used to guide a tissue anchor delivery device through the use of an anchor guide slidably coupled to at least one semi-rigid wire. It may be advantageous for the anchor guide to be rotationally fixed relative to at least one wire, for example by coupling the anchor guide to two or more substantially parallel semi-rigid wires. Rotational fixation can also be achieved by coupling the anchor guide to at least one semi-rigid wire having a non-circular cross sectional shape, for example triangular, square, rectangular, pentagonal, hexagonal, and the like. Alternately, rotational fixation can be achieved by taking advantage of the curvature of the semi-rigid coupled wire, by making the coupler long enough to have three non-linear points of contact with the curved wire.

In an additional aspect, the tissue anchor is turned to anchor to the tissue via a flexible torque wire that is capable of supporting sufficient torque to screw the anchor into the tissue and, if necessary, to unscrew the anchor from the tissue and remove it. A laser cut spiral can be used to make the tube flexible, but such a spiral exhibits substantial torque strength asymmetry, supporting more torque in one direction (for example, clockwise) than in the opposite direction (for example, counter-clockwise). Self-interlocking features on the spiral can reduce this torque strength asymmetry by preventing relative motion of the adjacent turns of the spiral. Cut shapes such as zig-zags, teeth, offset spiral cuts and the like can create self-interlocking features. Alternately, cutting the spiral off-axis, or at an angle to the axis, can also create self-interlocking geometry within the spiral cut. Cut shapes can be combined with off axis cuts and/or cuts at an angle to the axis to further improve torque resistance of the flexible torque wire.

In further examples, the probe elements may contain radiopaque material(s), for example in the form or strips, layers, features, patterns, and the like, to enhance their fluoroscopic visibility. In still further examples, the probe elements may contain echogenic features to improve their visibility to ultrasonic imaging techniques. For example, the echogenic features may include one or more of the following: retroreflective surface textures, air bubbles, hollow glass beads, closed cell foam structures, or a mix of materials with significantly different stiffnesses. In preferred examples, the probe elements will incorporate both radiopaque materials and echogenic features.

In some examples, the probe elements are attached to a sheath. In other examples, the probe elements are attached to a therapeutic, diagnostic, locating/positioning or marking device that passes through a sheath. In other examples, the probe elements are attached to an implantable device. In further examples, the implantable device is a helical anchor that couples with a target tissue. In still further examples, the probe elements are configured and arranged to hold or stabilize the implantable device in apposition to the tissue while the tissue heals after implantation. In still further examples, the probe elements are configured and arranged to be held in apposition to the tissue by the implantable device while the tissue heals after implantation.

In some examples, the target tissue comprises a mitral valve annulus. In other examples, the target tissue comprises an aortic valve annulus. In still other examples, the target tissue comprises a tricuspid valve annulus. In yet other examples, the target tissue comprises a pulmonary valve annulus, and in further examples, the target tissue comprises one or more venous valves.

The probe elements may interact with the target tissue in a variety of ways, for example by deflecting in response to engaging the target tissue. In other examples, the probe elements interaction may comprise electrically contacting, coupling, or sensing with the target tissue. In still other examples, the probe elements may be configured to vibrate, oscillate, or otherwise move when out of tissue contact, e.g. in response to blood or other fluid flow, an applied current, or other stimulation. In such cases, tissue contact can be detected when the probe elements stop moving when in response to tissue contact.

In additional examples, the probe elements are attached to the elongate device and configured to interact with the target tissue in a manner which indicate distances between the target tissue and a location on the elongate device. In a further example, the probe elements interact with the target tissue differently as the distance between the elongate device and the probe elements is increased or decreased. In other examples, different individual probe elements or sets of probe elements interact differently with the target tissue depending on the distance between the target tissue and the elongate device, e.g. longer probe elements may deflect in response to engaging tissue sooner than shorter probe elements; probe elements oriented at particular angles relative to the elongated device may deflect in response to engaging tissue sooner than other probe elements; probe elements having different shapes (linear, non-linear, sinusoidal, bifurcated, trifurcated, etc.) may deflect in response to engaging tissue at times different than shorter probe elements.

In some examples, the elongate device with probe elements may traverse the venous system. In other examples, the elongate device with probe elements may traverse the inferior vena cava. In a further example, the elongate device with probe elements crosses the septum between the right atrium and the left atrium. In still further examples, the elongate device with probe elements crosses the septum between the right atrium and the left atrium in the region of the fossa ovalis.

In some examples, the elongate device with probe elements traverses the arterial system. In further examples, the elongate device with probe elements traverses the aorta. In yet further examples, the elongate device with probe elements enters the left ventricle. In still further examples, the elongate device with probe elements cross from the left ventricle to the left atrium. In other examples, the elongate device with probe elements crosses from the left ventricle to the left atrium between the leaflets of the mitral valve.

In a first aspect, the present invention provides apparatus in the form of a surgical locating tool. The surgical locating tool may be used in a variety of surgical procedures, particularly less-invasive and percutaneous surgical procedures where visual access is limited, typically relying on fluoroscopy, ultrasound, optical coherence tomography (OCT), optical cameras, and the like. The surgical locating tools of the present invention can provide positional feedback as the locating tool approaches and engages a target location on a patient tissue site, often where the target location cannot be adequately visualized using external visioning capabilities. In particular, the positional feedback may be provided by one or more probe elements on the surgical locating tool, as will be described in more detail below.

An exemplary surgical locating tool constructed in accordance with the principles of the present invention comprises a shaft having one or more probe elements coupled thereto. The shaft typically has an engagement end, and the shaft will usually be configured to deliver and implant or to engage in interventional tool against an internal tissue surface. The one or more probe elements may be coupled or otherwise configured to extend outwardly from the engagement end of the shaft, and the probe elements are typically configured to detectably deflect when engaged against or in proximity with the internal tissue surface.

In some instances, the probe elements may be configured to be imaged by medical imaging devices, including any of fluoroscopic, ultrasonic, OCT, or other optical imaging systems of type commonly employed in performing such less-invasive or percutaneous surgical procedures. More specifically, the probe elements may be radiopaque, typically incorporating or attached to radiopaque markers, so that they are imageable under fluoroscopy. Alternatively, the probe elements may be acoustically opaque to enhance imaging under ultrasound observation. In still other instances, the probe elements may be optically visible using optical imaging sensors, such as viewing by cameras, CCD's, and the like, placed on other devices proximate the issue target sites. In still further instances, deflection may be detected by sensors attached to the probe elements, such as stress sensors, strain sensors, position encoders, and the like.

In still other instances, the shaft of the surgical locating tool of the present invention will be configured to deliver an implant to the target tissue site. For example, the shaft may have a channel which extends or opens to the engagement end of the shaft. The channel may be a receptacle or other cavity which extends only part way through or into the shaft. In most instances, however, the channel will extend an entire length of the shaft so that an implant may be delivered through the shaft after the engagement end has been located adjacent the target surgical site.

In yet other instances, the channels or other features of the shaft may be configured to position an interventional tool, such as an electrosurgical device, a tissue ablation device, a tissue resection device, or the like. In such instances, the shaft may be configured to position a separate interventional tool or, alternatively, the shaft may itself incorporate the interventional tool, i.e., the interventional tool or device may be integrated with locating tool to incorporate an interventional capability.

In certain instances, the locating tools of the present invention will have a plurality of probe elements at the engagement end of the shaft, typically from 2 to 24 probe elements. The probe elements may be arranged symmetrically or asymmetrically about an axial center line of the shaft. The probe elements may have the same or different lengths. The probe elements may have the same or different shapes. The probe elements may be arranged collectively to taper radially outwardly in a direction away from the engagement end of the shaft, e.g., may be arranged in a generally conical configuration with a large end of the cone spaced away from the engagement end of the shaft. In still other instances, the probe element(s) may be oriented at the same or different angles relative to the axial center line of the shaft, where the angles may vary from proximal end or section of the probe element in a direction toward a distal end or section of the probe element. The probe elements may have a constant cross-sectional area or shape or may have cross-sectional areas or shapes which vary along their lengths. Another instances, the probe elements may be configured to deflect primarily at a base end where they are attached to the shaft or may be configured to have a distributed deflection along their lengths.

In a second aspect, the present invention provides methods for locating and modifying an internal tissue surface of a patient. The methods comprise engaging one or more probe elements on or near an engagement end of a shaft against a target location on the internal tissue surface. Deflection of one or more of the probe elements is then observed to determine a position of the engagement end of the shaft relative to the target location. A tissue-modifying event may then be initiated when the engagement end of the shaft is at a desired position relative to the target location on the tissue.

In specific instances, observing the probe elements may comprise at least one of fluoroscopic imaging, ultrasonic imaging, and optical imaging. In the case of fluoroscopic imaging, the one or more probe elements may be radiopaque, partially radiopaque, or include radiopaque elements or markers disposed along the length of the probe element. In the case of ultrasonic imaging, the one or more probe elements may be acoustically opaque, reflective, or echogenic. In the case of optical imaging, the probe elements may be imaged by a camera on a tool near the location of the target tissue. In other instances, the probe elements may be imaged by OCT, or other surgical imaging methods. As an alternative to imaging, deflection of the probe elements may be detected using motion sensors attached or coupled to the probe elements, such as stress transducers, strain transducers, position encoders, and the like.

Initiating a tissue-modifying event may comprise delivering an implant from the shaft to, tissue at or near the target location. For example, the implant may comprise a plication tip or other element intended to be implanted on a heart valve annulus, such as a mitral valve annulus.

In other instances, initiating the tissue-modifying event may comprise positioning the shaft to engage an interventional tool against the target tissue. For example, the interventional tool may be advanced through the shaft to a position near the engagement end of the tool which is being held proximate the target tissue location. In other instances, the interventional tool may be integrated with the shaft of the locating tool.

In further instances of the methods of the present invention, a plurality of probe elements will be engaged against the target location on the internal tool surface. The plurality may comprise two or more probe elements, typically being in a range from 2 to 24 probe elements. The probe elements may be arranged symmetrically or asymmetrically about a center line of the shaft. The probe elements may all have the same length or may have different lengths. The probe elements may comprise longer probe elements and shorter probe elements, typically being interdigitated or otherwise interspersed with each other to engage tissue at different times or at different positional locations of the shaft. The probe elements may taper radially outwardly in a distal direction away from the engagement end of the shaft, for example in a radially outward conical pattern. The shapes of the probe elements may vary, including linear, nonlinear, serpentine, and the like. The probe elements may deflect radially outwardly from a center line of the probe shaft at similar angles or different angles. The probe elements may have a constant cross-sectional shape or area, or the cross-shape or area may vary among different individual or groups of probe elements. The probe elements may be configured to deflect primarily at a base end where they are attached to the shaft, e.g., the base end may act as a pivot or fulcrum for deflection of the probe element. In other instances, the probe elements may be flexible along their lengths and configured to deflect in a distributed manner between the base and detached to the shaft and a distal end in free space.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an elongate device with probing elements extending distally and outward. Depending on the width of the probe elements, they can be considered flaps that have greater stiffness and will be considered as probe elements hereafter.

FIG. 2 shows an end view of the elongate device with probing elements and an integral central channel

FIG. 3 shows an elongate device with probing elements extending distally in line with the device.

FIG. 4 shows a side view of the elongate device in FIG. 1.

FIG. 5 shows the elongate device of FIG. 1 approaching a tissue wall at an angle, the tissue wall having a movable tissue segment, for example, a valve leaflet.

FIG. 6 shows the elongate device of FIG. 5 in contact with the wall, with the probing elements deflected and in contact with the target tissues.

FIG. 7 shows the elongate device of FIG. 6, with the probing elements remaining in contact with the movable tissue segment as it moves

FIG. 8 shows an end view of an elongate device having probe elements of varying lengths.

FIG. 9 shows a side view of an elongate device having probe elements extending primarily outward from the elongate device.

FIG. 10 shows an end view of an elongate device having probe elements with variable cross section. As shown, the section is thinner and therefore more flexible near the elongate device, creating a hinge effect.

FIG. 11 shows an end view of an elongate device having probe elements with variable cross section. As shown, the section changes in thickness toward the tips of the probe elements, creating a gentler, more flexible tip on one or more probe elements.

FIG. 12 shows an end view of an elongate device having probe elements connected at the ends by bridging segments.

FIG. 13 shows an end view of an elongate device having probe elements with a branching structure on at least some of the probe elements.

FIG. 14 shows a side view of an elongate device having probe elements with an angle relative to the elongate device that changes along the length of the probe element.

FIG. 15 shows a side view of an elongate device having probe elements which branch to create a probe segment directed inward and distally.

FIG. 16 shows a side view of an elongate device having probe elements which branch to create a probe segment directed inward and proximally.

FIG. 17 shows two adjacent connected probe elements, the connection having a shape that allows the connection to partially fold so that the connection and the probe elements can move inwardly to a smaller diameter.

FIG. 18 shows an elongate device with probe elements in section view, with an anchoring device through the center channel. The anchoring device is coupled to the target tissue.

FIG. 19 shows an elongate device with probe elements, and anchoring device coupled to the tissue, and a tissue shaping template coupled to the anchoring device.

FIG. 20 shows an elongate device with an array of probe elements arranged along its length.

FIG. 21 shows an elongate device with probe elements having a solid center support.

FIG. 22 shows a number of alternate cross sections for the elongate device.

FIGS. 23A-23B show an elongate device with probe elements, the elongate device having an internal structure that allows the height or diameter of the device to change by moving one member proximally or distally relative to the other member.

FIGS. 24A-24B show an elongate device having probe elements that connect at the distal end to form a basket. Moving one end of the basket proximally or distally relative to the other end adjusts the diameter of the basket.

FIG. 25 shows a simplified elongate device having two probe elements, the elongate device configured to rotate about an axis to change the orientation of the two probe elements relative to the target tissue.

FIG. 26 shows an elongate device having two probe elements composed of at least two distinct materials.

FIGS. 27A-27B show an elongate device having probe elements and an outer sheath. Moving the probe elements proximally or distally relative to the outer sheath adjusts the effective length of the probe elements.

FIGS. 28A-28C show a system of nested elongate devices with probe elements of different lengths, and a tissue coupling anchor to be delivered to the target tissue.

FIG. 29 shows an elongate device with multiple independent probe elements, one or more of which can be moved proximally and distally relative to one or more of the others.

FIG. 30 shows a catheter for delivering a tissue anchor, the catheter including one or more arms that interact with the atrial and or ventricular walls to guide the tissue anchor to the valve

FIG. 31A-31C show a device for delivering a tissue anchor consisting of a plurality of flexible wires that interact with the ventricular wall, the atrial wall, the valve leaflets, and/or the valve annulus to guide the tissue anchor to the valve annulus.

FIG. 32 shows an anchor guide that slides along a curved wire, while preventing axial rotation of the anchor guide relative to the curved wire.

FIG. 33 shows a device for delivering a tissue anchor including a plurality of flexible arms, which interact with the tissue surrounding the valve annulus to guide the tissue anchor to the valve annulus.

FIG. 34 shows an alternative embodiment of the device of FIG. 33, where the flexible arms at least partially surround at least a portion of the anchor to reduce the dimetral and/or length space required for the delivery device.

FIG. 35 shows a system of catheters using magnets to position an anchor delivery catheter relative to a valve annulus.

FIGS. 36A-36B show an alternative anchor delivery catheter which is delivered through a guide sheath with the magnet distal to the anchor. The magnet is movable to second position alongside the delivery catheter, which aligns the delivery catheter relative to a magnet placed on the opposite side of the valve in order to guide the anchor to the valve annulus.

FIG. 37 shows a cylindrical magnet polarized in the axial direction.

FIG. 38 shows a cylindrical magnet polarized in a radial direction.

FIG. 39 shows a ring magnet polarized in the axial direction.

FIG. 40 shows a ring magnet polarized in a radial direction.

FIG. 41 shows a ring magnet with multiple magnetic poles arranged around the perimeter.

FIG. 42 shows a helical torque application tube with a self-locking spiral cut.

FIGS. 43A-43H show a magnetic anchor placement system comprising a target magnet catheter and an anchor delivery catheter with a radially polarized semi-circular anchor magnet in place adjacent a heart valve.

FIG. 44 is a graphical illustration of the left atrium and mitral valve of a heart with one magnetic catheter in place in the ventricle beneath the posterior mitral annulus, and a second magnet catheter in place in the atrium above the posterior mitral annulus. Magnetic attraction aligns the two catheters.

FIG. 45 shows the left atrium and mitral valve of a heart with one magnetic catheter in place in the ventricle beneath the posterior mitral annulus, and a second magnet catheter in place in the atrium above the posterior mitral annulus, the two magnetic components of the catheters having axial polarity, with opposite poles at their distal ends, creating magnetic attraction between the two catheters.

FIG. 46 shows the left atrium and mitral valve of a heart with one magnetic catheter in place in the ventricle beneath the posterior mitral annulus, and an anchor delivery catheter in place in the atrium above the posterior mitral annulus, the atrial catheter having a tip made of non-magnetized but ferro-magnetic material, creating magnetic attraction between the two catheters. The atrial catheter is shown delivering a helical coil tissue anchor to the mitral annulus.

FIG. 47 shows the left atrium and mitral valve of a heart with one magnetic catheter in place in the ventricle beneath the posterior mitral annulus, and a second magnet catheter in place in the atrium above the posterior mitral annulus, magnetic component of the ventricular catheter having axial polarity and the magnetic component of the atrial magnet having radial polarity, and rotated so that the pole opposite the distalmost pole of the ventricular magnet is oriented toward the ventricular magnet, creating magnetic attraction between the two catheters.

FIGS. 48A and 48B show the left atrium and mitral valve of a heart with one magnetic catheter in place in the ventricle beneath the posterior mitral annulus and a second magnet catheter in place in the atrium above the posterior mitral annulus. The magnetic component of the locating (ventricular) catheter has an axial polarity (as indicated by the arrow), and the magnetic component of the anchor delivery (atrial) catheter is coupled to one side only of the anchor delivery catheter and has a radial polarity (as indicated by the arrow), where the pole of each magnet is oriented to allow the anchor delivery catheter to be rotated to bring opposite magnet poles into alignment, magnetically coupling the distal ends of the catheters across the annulus. FIG. 48B is a detailed view of the of the distal ends of the catheters illustrating advancement of a helical anchor from the anchor delivery catheter along a path defined by the orientation of the distal end of the anchor delivery catheter into the valve annulus.

FIG. 49 shows a catheter with a magnetic tip, the magnetic tip being completely closed. The catheter proximal to the magnetic tip may be flexible to follow curved anatomy or may be actively steerable to a desired position.

FIG. 50 shows a catheter with a magnetic tip, the magnetic tip having a lumen which communicates with the central lumen of the catheter. This lumen may allow the catheter to be positioned over a guidewire, or may allow delivery of an anchor, a guidewire, contrast media, or other desirable material or devices to the target anatomy. The catheter proximal to the magnetic tip may be flexible to follow curved anatomy or may be actively steerable to a desired position.

FIG. 51 shows a catheter with a magnetic tip placed adjacent a valve annulus in a heart chamber. As shown, two arms extend from the magnet, the arms being constructed to be echogenic, radiopaque, or a combination of the two. The arms thus extended create an image reference for rotational alignment of an implant placed in the adjacent chamber of the heart.

FIG. 52 shows a catheter with a magnetic tip placed adjacent a valve annulus in a heart chamber. As shown, two arms extend from the magnet, the arms being constructed to be echogenic, radiopaque, or a combination of the two. The arms are connected at the distal end by a stationary -ring at least partially surrounding the magnet and are connected at the proximal end by a movable ring at least partially around the catheter. The proximal ends of the arms are connected to at least one actuator (not shown), either directly or via the movable ring. Moving the actuator causes the arms to either move closer to the magnet, for example for insertion or removal from the heart chamber, or to extend farther from the magnet for improved visualization. The arms thus extended create an image reference for rotational alignment of an implant placed in the adjacent chamber of the heart.

FIG. 53 shows a catheter with a magnetic tip, the magnetic tip having a lumen which communicates with a lumen of the catheter. One or more guidewires extend through this lumen and curves to one or more sides. Imaging the guidewire or guidewires indicates the device alignment with one or more spaces in the adjacent anatomy, and thus creates an image reference for rotational alignment of an implant placed in a nearby anatomic location.

FIG. 54 shows a catheter with a magnetic tip, the magnetic tip having a lumen which communicates with the central lumen of the catheter. A guidewire extends through this lumen, the guidewire having an expandable pair of arms. As shown, two arms extend from the guidewire, the arms being constructed to be echogenic, radiopaque, or a combination of the two. The arms are connected at the distal end by a stationary ring at least partially surrounding the core of the guidewire and are connected at the proximal end by a movable ring at least partially around the core of the guidewire. The proximal ends of the arms are connected to an outer sleeve of the guidewire, either directly or via the movable ring. Moving the outer coil of the guidewire relative to the core of the guidewire causes the arms to either move closer to the core of the guidewire, for example for insertion or removal from the magnet catheter, or to extend farther from the core of the guidewire for improved visualization. The arms thus extended create an image reference for rotational alignment of an implant placed in adjacent anatomy.

FIG. 55 shows a catheter with a primary shaft, an accessory lumen, and a magnetic tip, the magnetic tip having a lumen which communicates with the accessory lumen. This accessory lumen may allow the catheter to be positioned over a guidewire, or may allow delivery of an anchor, a guidewire, contrast media, or other desirable material or devices to the target anatomy. The magnetic tip is coupled to the primary shaft, which passes at least partially through the lumen of the magnetic tip. The primary shaft may flexible to follow curved anatomy or may be actively steerable to a desired position.

FIG. 56 shows a catheter with a primary shaft, an accessory lumen, and a magnetic tip, the magnetic tip having a partial lumen coupled with the accessory lumen. This accessory lumen may allow the catheter to be positioned over a guidewire, or may allow delivery of an anchor, a guidewire, contrast media, or other desirable material or devices to the target anatomy. The magnetic tip is coupled to the primary shaft, which passes at least partially through the lumen of the magnetic tip. The primary shaft may flexible to follow curved anatomy or may be actively steerable to a desired position.

FIG. 57 shows a catheter with a magnetic tip, the magnetic tip having a lumen which communicates with a lumen of the catheter from a side port proximal to the catheter tip. One or more guidewires extend through this lumen and curves to one or more sides. Imaging the guidewire or guidewires indicates the device alignment with one or more spaces in the adjacent anatomy, and thus creates an image reference for rotational alignment of an implant placed in a nearby anatomic location.

### DETAILED DESCRIPTION OF THE INVENTION

The phrase “valve annulus” as used herein and in the claims means a ring-like tissue structure surrounding the opening at base of a heart valve that supports the valve's leaflets. For example, the annulus of the mitral valve, the tricuspid valve, the aortic valve, the pulmonary valve, venous valves and other annuluses of valves in the body. In the mitral valve, the annulus typically is a saddle-shaped structure that supports the leaflets of the mitral valve.

The phrase “peripheral wall” as used herein and in the claims as applied to a valve annulus means a surface or portion of the tissue of the valve annulus, and/or a portion of the tissue adjacent to the valve annulus.

As used herein and in the claims, an “implant” means an article or device that is introduced into and left in place in a patient's body by surgical methods, including open surgery, intravascular surgical methods, percutaneous surgical methods, and least invasive or other methods. For example, aortic valve replacement implant, coronary stent implant, or other types of implants.

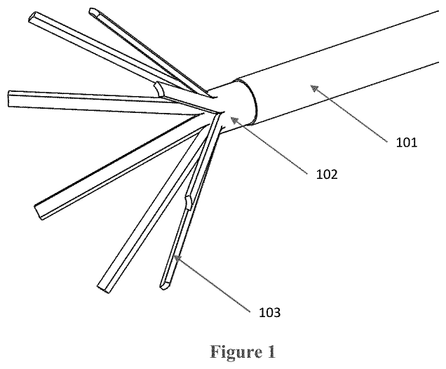

As shown in FIG. 1, an elongate device 101 has a probe element junction 102 extending into one or more probe elements 103. In FIG. 1, eight probe elements 103 extend distally and outward from the elongate device 101, however other shapes and numbers of probe elements may be advantageous.

Probe elements can be formed by, for example, by insert molding one or more probe elements together and attaching to the elongate device, by laser cutting a tube formed of probe element material, or by cutting the desired probe element pattern in a flat and shaping it (if needed) to fit the elongate device, by photochemical etching, or a combination of these processes. The probe elements can be shaped during processing (particularly in the case of the injection molded variants), bent to shape after cutting, or heat set to final shape in post processing. Additional features, such as hinge points, sensors, conductive pads, or wires, can be attached by processes including bonding, welding, crimping, and the like.

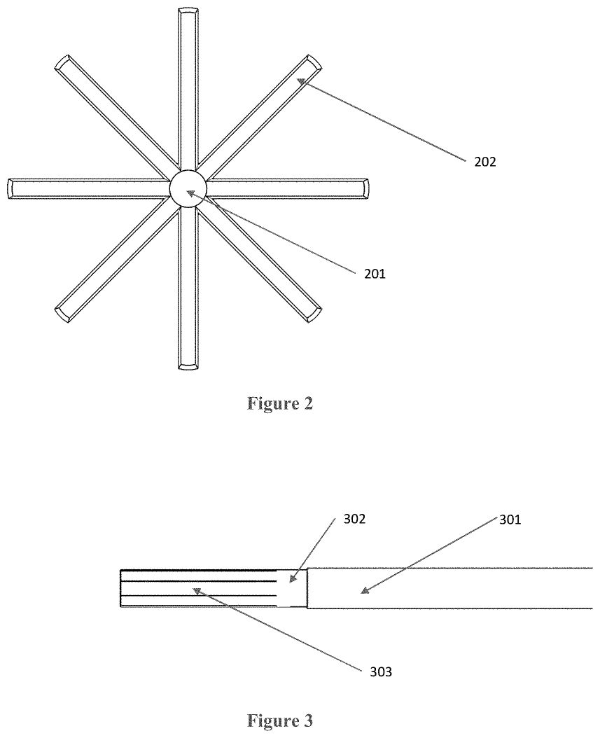

FIG. 2 shows an end view of the elongate device 101 from FIG. 1, showing the inner surface 202 of the probe elements, and a central channel 201. The central channel 201 may be used for delivery of a therapeutic or diagnostic device or material to a target tissue site. An implant may be placed through the central channel 201. The central channel 201 may also be used to place a marker into the tissue, or inject a contrast solution for imaging tissue, lumens, or body cavities adjacent to the probe elements. The central channel 201 may also be used to biopsy or remove target tissue.

FIG. 3 shows an elongate device 301 having a probe element junction 302 and one or more probe elements 303 extending distally in the direction of the elongate device 301. Probe elements 303 in this configuration are deliverable through a channel the same size as the elongate device 301. It may be advantageous to form probe elements 303 in this configuration, or to temporarily constrain outwardly directed probe elements (for example, probe elements 202 shown in FIG. 2) in this configuration for delivery to the target tissue site.

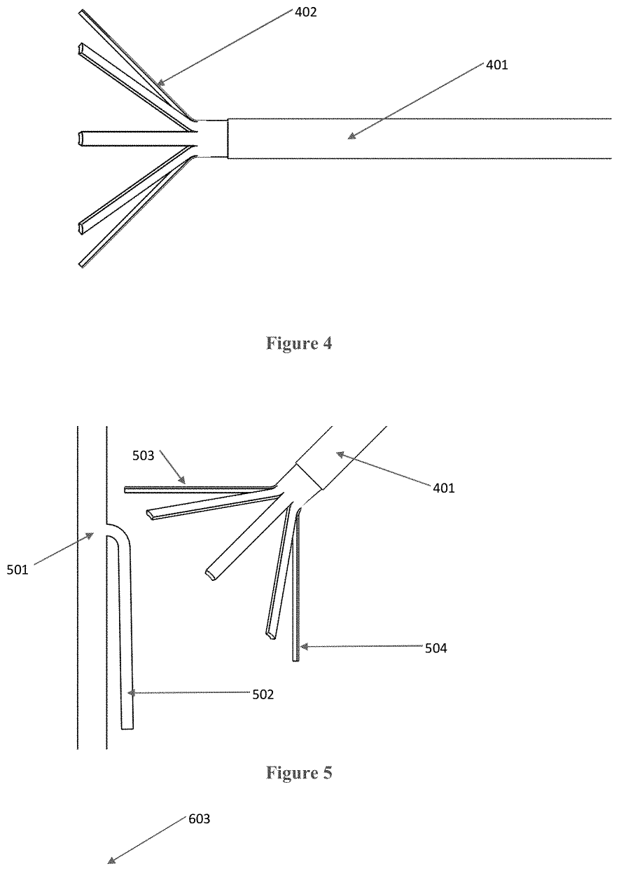

FIG. 4 shows a side view of an elongate device 401 having one or more probe elements 402 which extend outward and distally from the elongate device at an angle of approximately 45 degrees.

FIG. 5 shows the elongate device 401 of FIG. 4 approaching target tissue 501 at approximately a 45 degree angle. At this angle, the top probe element 503 approaches target tissue at approximately a right angle, and the bottom probe element 504 is approximately parallel to the target tissue. A mobile segment 502 of the target tissue is approximately parallel to the target tissue.

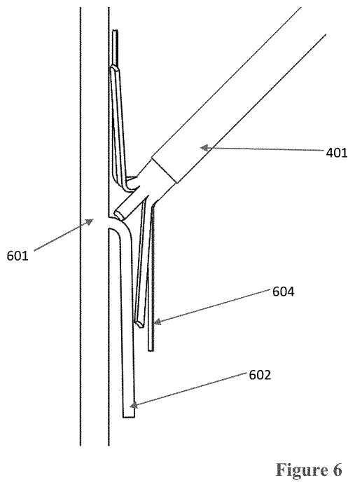

FIG. 6 shows the elongate device 401 of FIG. 4 approximating target tissue 601 at approximately a 45 degree angle. When elongate device 401 and target tissue 601 are held in approximation, the top probe element 603 deflects to extend upward approximately parallel to the target tissue, and the bottom probe element 604 remains directed downward approximately parallel to the target tissue. A mobile segment 602 of the target tissue is approximately parallel to the target tissue.

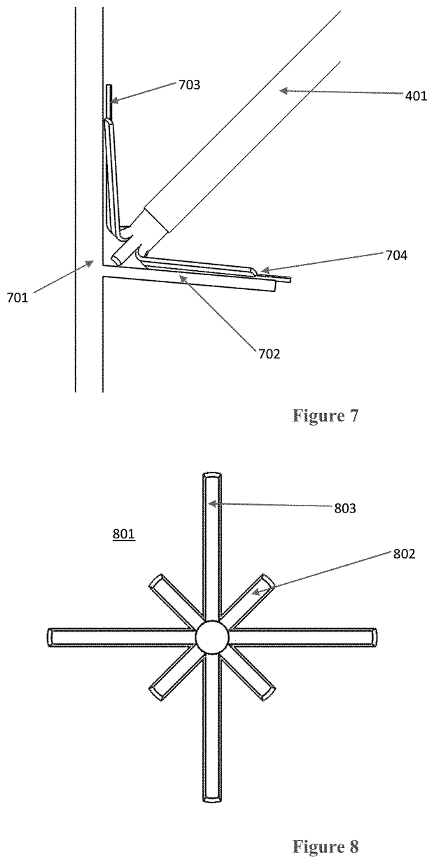

FIG. 7 shows the elongate device 401 of FIG. 4 approximating target tissue 701 at approximately a 45 degree angle. In this figure, the mobile segment 702 of the target tissue has moved to a position approximately perpendicular to the target tissue 701. When elongate device 401 and target tissue 701 are held in approximation, the top probe element 703 remains deflected upward approximately parallel to the target tissue, and the bottom probe element 704 deflects along with the mobile tissue segment 702. In this configuration, images showing the motion of the probe element 704 can be used to infer motion of the mobile segment 702 of the target tissue. Images showing the motion of the probe element 704 can also be used to infer the location of the elongate device 401 relative to the mobile segment 702 of the target tissue.

FIG. 8 shows and end view of an elongate device 801 having one or more short probe elements 802 and one or more long probe elements 803A-B. In use, the long probe elements 803A-B will move with moving tissue at a first distance (shown by line 804) from the elongate device 801, while the short probe elements 802 will not be affected by tissue motion at said first distance. When the elongate device 801 is moved closer to the moving tissue, to a second distance (shown by line 805) that is less than said first distance, both the short probe elements 802 and the long probe elements 803A and 803B will be affected by tissue motion. Similarly, probe elements of 3 different lengths, 4 different lengths, or more could be used to indicate position of the elongate device relative to moving tissue. Probe elements can interact with stationary tissue features (for example, a luminal opening in a wall) in a similar fashion, with long probe elements 803 reacting first to the stationary tissue feature, and short probe elements 802 reacting only when the elongate device 801 is moved closer to the stationary tissue feature.

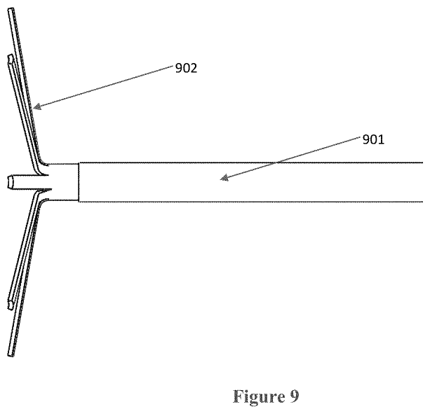

FIG. 9 shows an end view of an elongate device 901 having one or more probe elements 902 extending outwardly from the elongate device 901. The angle between the probe elements 902 and the long axis of the elongate device 901 approaches perpendicular. In this configuration, the probe elements 902 would move in response to bumps or curves in the surface of a target tissue.

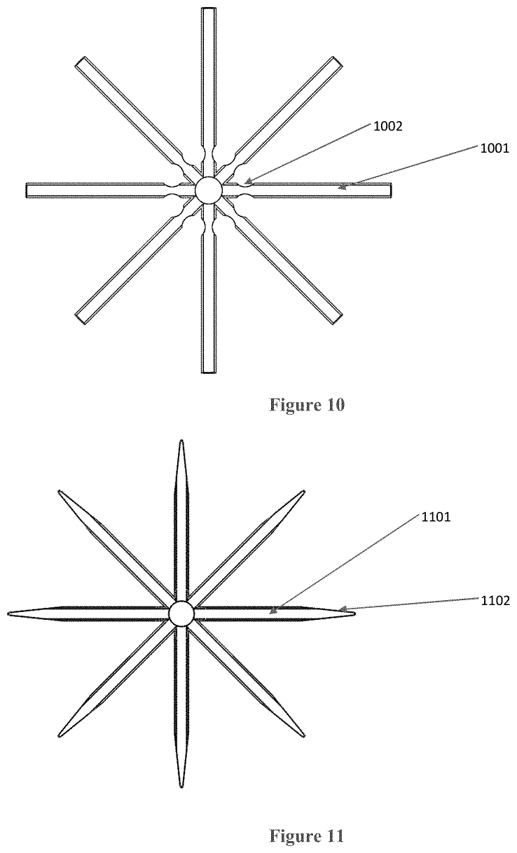

FIG. 10 shows an end view of an elongate device having one or more probe elements 1001 with a varying cross section. As shown, there is a reduced cross section 1002 near the junction of the probe elements 1001 and the elongate device. This reduced cross section 1002 creates a more flexible “hinge” region, offering increased control over the shape the probe elements 1001 take when interacting with target tissue.

FIG. 11 shows an end view of an elongate device having one or more probe elements 1101 with a varying cross section. As shown, there is a reduced cross section 1102 near the distal end of the probe elements 1101. This reduced cross section 1102 creates a more flexible tip region, offering increased control over the shape the probe elements 1101 take when interacting with target tissue.

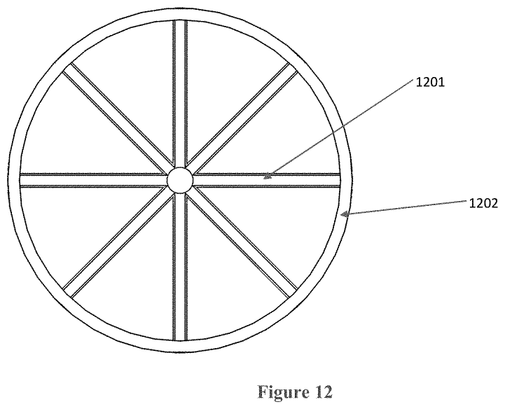

FIG. 12 shows an end view of an elongate device having two or more probe elements 1201 with one or more branches 1202 extending from one or more of the probe elements 1201 and connecting to one or more of the adjacent probe elements 1201. As shown, the branches 1202 extend from the ends of each of the eight probe elements 1201 and connect each of the adjacent probe elements 1201 to form a continuous ring. It may be advantageous to have the branches 1202 extend from a location proximal to the tip of the probe elements 1201, or to connect subsets of probe elements 1201, leaving others unconnected.

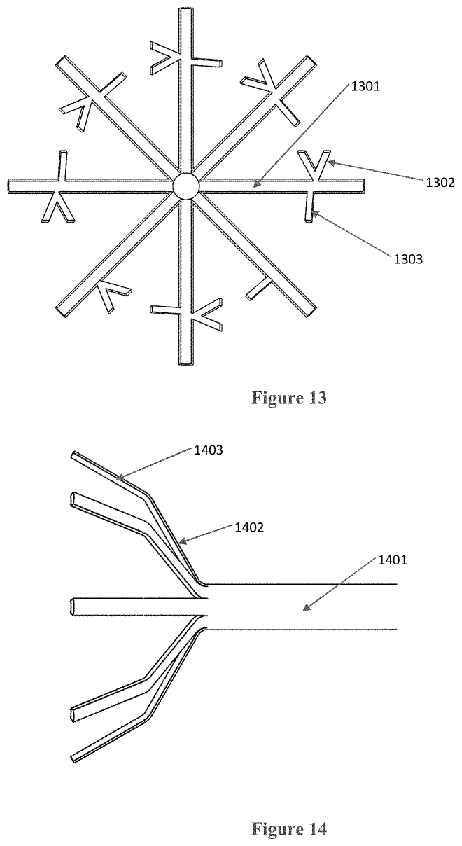

FIG. 13 shows an end view of an elongate device having one or more probe elements 1301 having forked branches 1302 and single branches 1303 extending from one or more of the probe elements. Each probe element 1301 can have no branches, one or more forked branches 1302, one or more single branches 1303, or a combination of forked branches 1302 and single branches 1303. The forked branches 1302 and single branches 1303 can extend substantially planar to the probe elements 1301 or be deflected inward or outward from the probe elements 1301 relative to the elongate device.

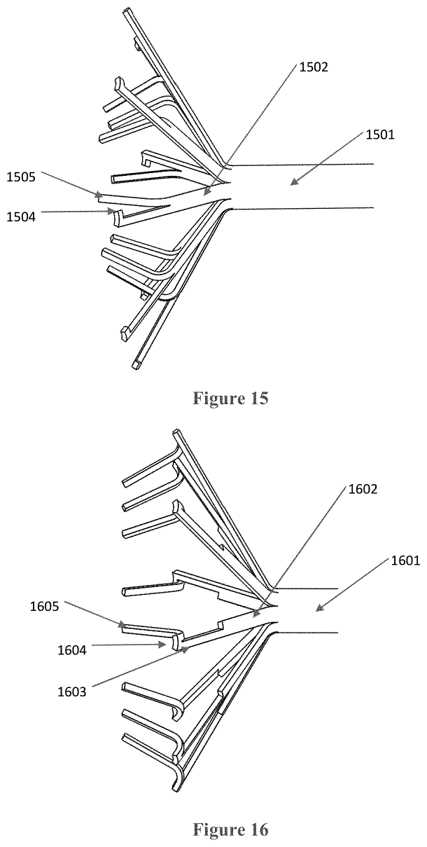

FIG. 14 shows an elongate device 1401 having one or more probe elements having a proximal segment 1402 extending from the elongate device 1401 at a first angle, and a distal segment 1403 of the probe elements extending from the proximal segment 1402 at a second angle relative to the long axis of the elongate device 1401. As shown, the second angle is a shallower angle relative to the elongate device 1401 compared to the first angle. Probe elements having proximal segments 1402 and distal segments 1403 at different angles will interact with target tissues in different ways than straight probe elements, offering different information about the location and motion of the target tissue than a straight probe element would. It may be further advantageous to combine both straight probe elements and probe elements having proximal segments 1402 and distal segments 1403 at different angles in the same elongate device.

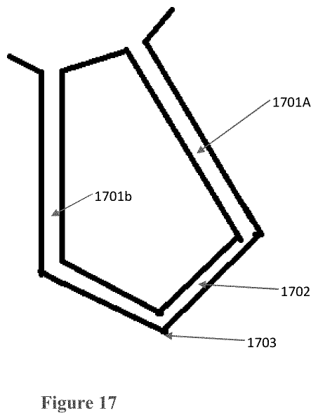

FIG. 15 shows a side view of an elongate device 1501 having one or more probe elements 1502 disposed at a first angle relative to the long axis of the elongate device 1501 with one or more branches 1505 disposed at a second angle relative to the long axis of the elongate device 1501. As shown, the branches 1505 extend distally and inwardly from the branching point. It may be advantageous for the branches 1505 to extend distally and outwardly from the branching point, or remain in substantially the same plane as the probe element 1502.

FIG. 16 shows a side view of an elongate device 1601 having one or more probe elements 1602 disposed at a first angle relative to the long axis of the elongate device 1601 with one or more branches 1605 disposed at a second angle relative to the long axis of the elongate device 1601. As shown, the branches 1605 extend proximally and inwardly from the branching point. It may be advantageous for one or more branches 1605 to extend proximally and outwardly from the branching point, or remain in substantially the same plane as the probe element 1602.

FIG. 17 shows two adjacent probe elements 1701A and 1701B, connected by a connecting branch 1702 having a bend 1703. The bend 1703 can fold as the probe elements 1701A and 1701B move relative to each other. In a small diameter delivery configuration, the connecting branch 1702 will be folded on itself at the bend 1703, and the probe elements 1701A and 1701B will approximate each other, allowing the assembly to pass through a catheter appropriately sized for access to the target body area.

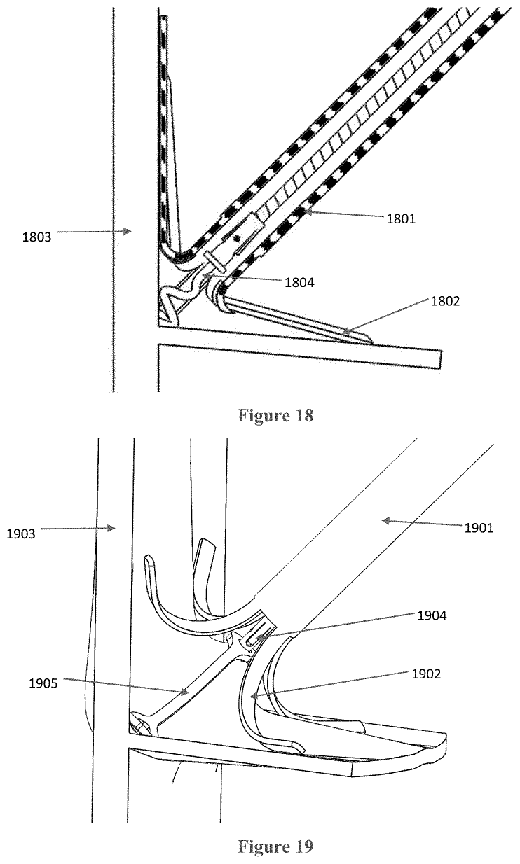

FIG. 18 shows a section view of an elongate device 1801 having one or more probe elements 1802 in contact with target tissue 1803. With the location of the elongate device 1801 relative to the target tissue 1803 verified by imaging methods aided by the one or more probe elements 1802, a tissue coupling anchor 1804 is placed through the center channel of the elongate device 1801 and coupled to the target tissue in the desired location. The probe elements 1802 in combination with various imaging modalities can be used to enhance visibility of the target tissue 1803 and aid in placing the tissue coupling anchor 1804 accurately relative to the target tissue 1803.

FIG. 19 shows an isometric view of an elongate device 1901 having one or more probe elements 1902 in contact with target tissue 1903. Disposed within the center channel of the elongate device 1901 is a tissue coupling anchor 1904. Coupled to the tissue coupling anchor is a tissue shaping template 1905, which reshapes the target tissue to a desired configuration. The probe elements 1902 in combination with various imaging modalities can be used to enhance visibility of the target tissue 1903 and aid in placing the tissue shaping template 1905 in the correct rotational orientation relative to the target tissue. After placement of the tissue shaping template 1905, the probe elements 1902 can be used to verify the re-shaping and motion of the tissue are within target parameters.

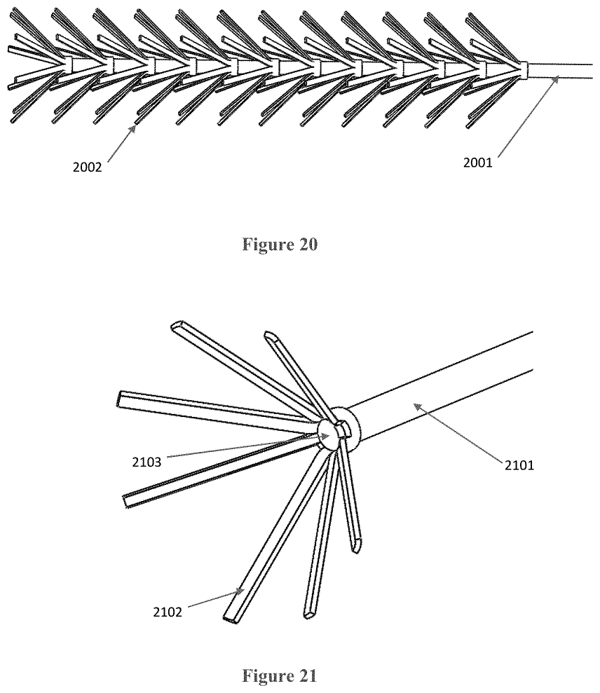

FIG. 20 shows an elongate device 2001 having an array of probe element groups 2002 disposed along its length. The displacement and motion of the individual probe element groups 2002 can be used to locate certain tissue features relative to the long axis of the elongate device 2001, or to locate more than one tissue feature relative to another tissue feature.

FIG. 21 shows an elongate device 2101 having one or more probe elements 2102 and a solid central cross section 2103. The cross section 2103 can be optimized to make the profile of the elongate device 2101 as small as possible to access small lumens, or to fit alongside other instruments.

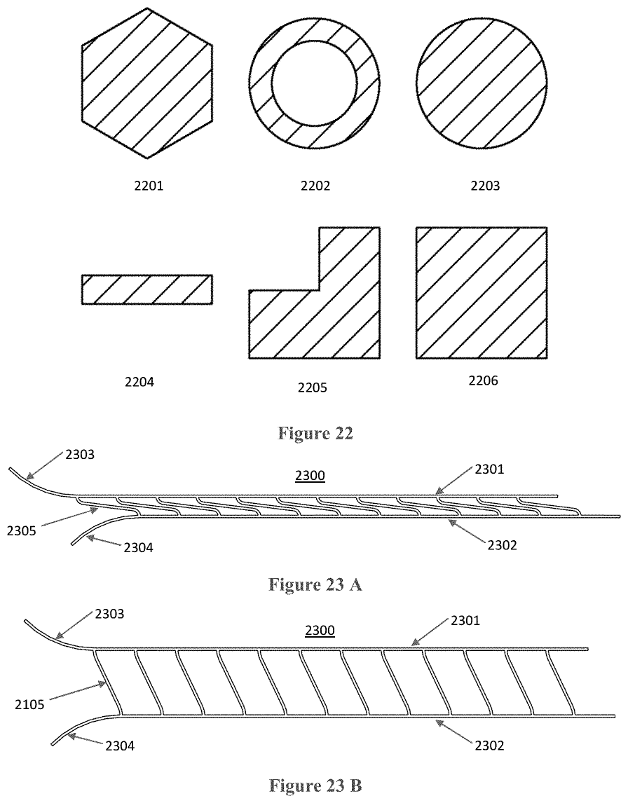

FIG. 22 shows a series of possible cross sections for an elongate device, including polygonal 2201, tubular 2202, circular 2203, flat 2204, cutout 2205, square 2206. Closely related shapes, for example polygons with different numbers of sides, tubes with multiple lumens, ellipses, arcuate segments, rectangles, etc. may also have advantages as cross sections for elongate devices.

FIG. 23 shows an elongate device 2300 with a variable height or diameter. The elongate device 2300 comprises a first elongate segment 2301 having one or more probe elements 2303 and a second elongate segment 2302 having one or more probe elements 2304, the two elongate segments 2301 and 2302 being connected by an array of rungs 2305 such that by moving the elongate segments 2301 and 2302 proximally or distally relative to each other, the separation distance between them, and therefore the height or diameter, can be change. FIG. 23 A shows this device in the narrow, small diameter, short configuration, and FIG. 23 B shows this device in the wide, large diameter, tall configuration.

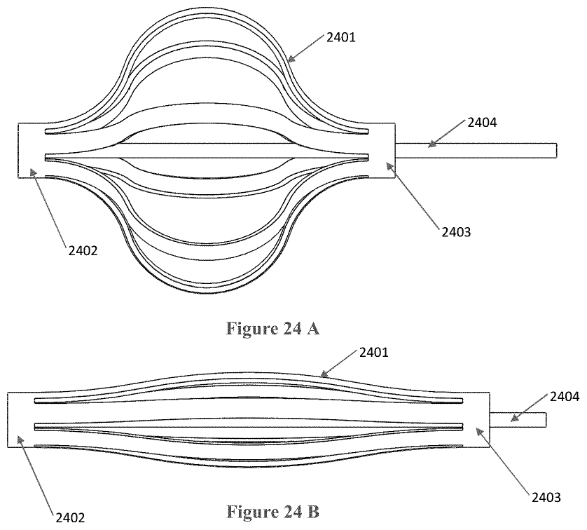

FIG. 24 shows an elongate device with one or more probe elements 2401 joined to a distal hub 2402 and a proximal hub 2403, the distal hub being coupled to a shaft 2404, and the proximal hub being slidably engaged to the shaft 2404. When the hubs 2402 and 2403 are brought closer together, the probe elements 2401 bend more and have an increased diameter (as shown in FIG. 24A). When the hubs 2402 and 2403 are moved farther apart, the probe elements 2401 straighten, and have a decreased diameter (as shown in FIG. 24B). This changeable diameter can be used to visualize the shape and size of body lumens, pockets, aneurysms, or other tissue features.

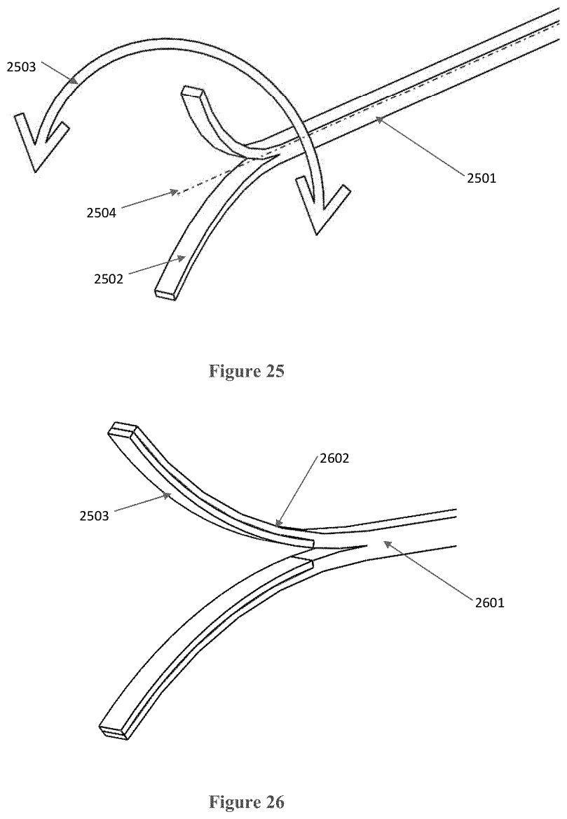

FIG. 25 shows an elongate device 2501 having one or more probe elements 2502 which can be rotated 2503 clockwise or counterclockwise around an axis 2504.

FIG. 26 shows an elongate device 2601 having one or more probe elements 2602 with a secondary feature 2503. The secondary feature can be a second material with enhanced imaging properties (for example, an echogenic layer), or a sensing element with a connection extending back through the elongate device 2601 (for example, an pressure sensor, a strain sensor, a piezoelectric material, microphone, oxygen sensor, electrode, or other similar sensing equipment). Such a sensor may offer additional information to the user, for example the blood oxygenation at the probe element 2602 could indicate if it is in the venous or arterial blood system.

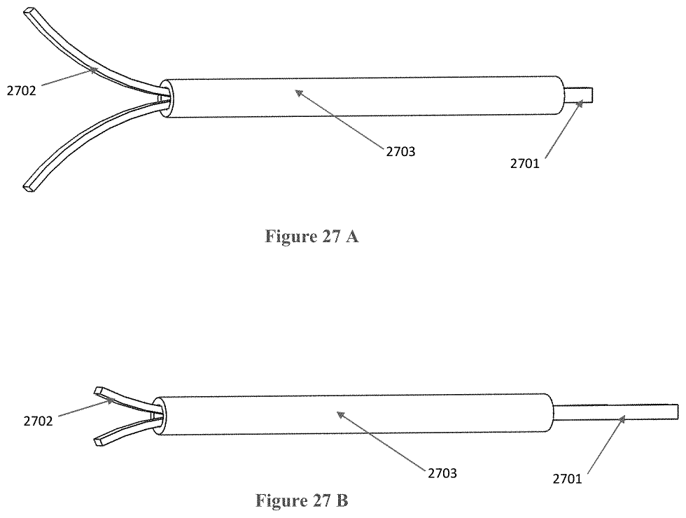

FIG. 27 shows an elongate device 2701 having one or more probe elements 2702 and a slidable sleeve 2703 disposed around the elongate device 2701.

FIG. 27A shows the slidable sleeve 2703 retracted proximally relative to the probe element 2702, with a first length of probe element 2702 extending distally from the slidable sleeve 2703. In this configuration, the probe elements 2702 could be used to guide the elongate device 2701 to the general vicinity of the target tissue.

FIG. 27B shows the slidable sleeve 2703 extended distally relative to the probe element 2702, with a second length of probe element 2702 extending distally from the slidable sleeve 2703. This second length is shorter than the first length illustrated in FIG. 27A. In this configuration, the probe elements 2702 could be used to guide the elongate device 2701 to the target tissue with greater precision than the configuration shown in FIG. 27A.

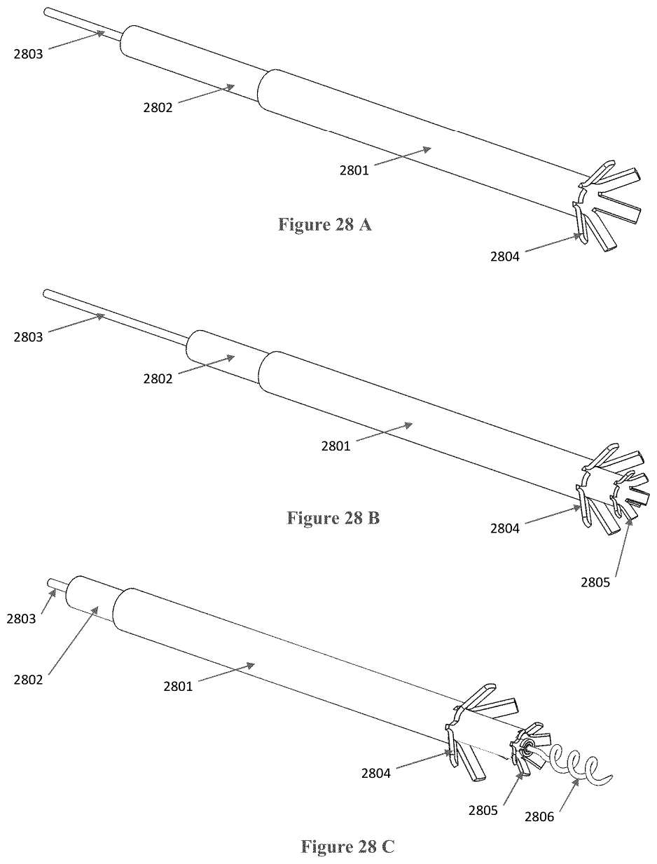

FIG. 28 shows an elongate device consisting of an outer sheath 2801, and inner sheath 2802 and an anchor shaft 2803 coupled to a tissue coupling anchor 2806.

FIG. 28A shows the device of FIG. 28 with the outer sheath 2801 covering both the distal end of the inner sheath 2802 and the tissue coupling anchor 2806. The outer sheath 2801 has one or more probe elements 2804 having a first length.

FIG. 28B shows the device of FIG. 28 with the inner sheath 2802 moved distally relative to the outer sheath 2801 so that the distal end of the inner sheath 2802 extends distally from the distal end of the outer sheath 2801. The inner sheath 2802 has inner probe elements 2805 having a second length on the distal end of the inner sheath. The second length of the inner probe elements 2805 is different than the probe elements 2804 of the outer sheath 2801. The different length elements can be used to resolve different size features. In addition, the longer probe elements can be used to approach the vicinity of the target tissue, and the shorter probe elements can be used to refine that positioning.

28C shows the tissue coupling anchor 2806 extending distally from both the inner sheath 2802 and the outer sheath 2803. In this configuration, the tissue coupling anchor can be coupled to the target tissue.

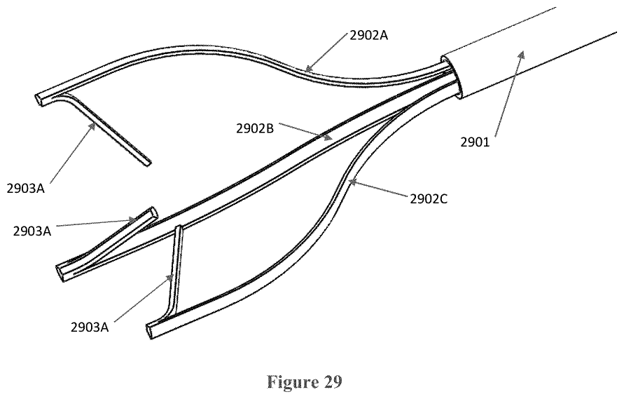

FIG. 29 shows an elongate device 2901 having at least one extensible probe elements 2902A, 2902B, or 2902C. At least one probe element 2902A can be extended or retracted proximally or distally relative to at least one other probe element 2902B. Probe element 2902A is shown with a branch 2903A, which can interact with stationary tissue, movable tissue, or fluid flow in a way that indicates the position of the branch relative to the target tissue. By adjusting the relative positions of two probe elements, 2902A and 2902B, the user can visualize a linear structure in the target tissue, for example a segment of a valve annulus. By adjusting the relative positions of three independent probe elements, 2902A, 2902B, and 2902C, the user can visualize a planar structure in the target tissue, for example a valve annulus.

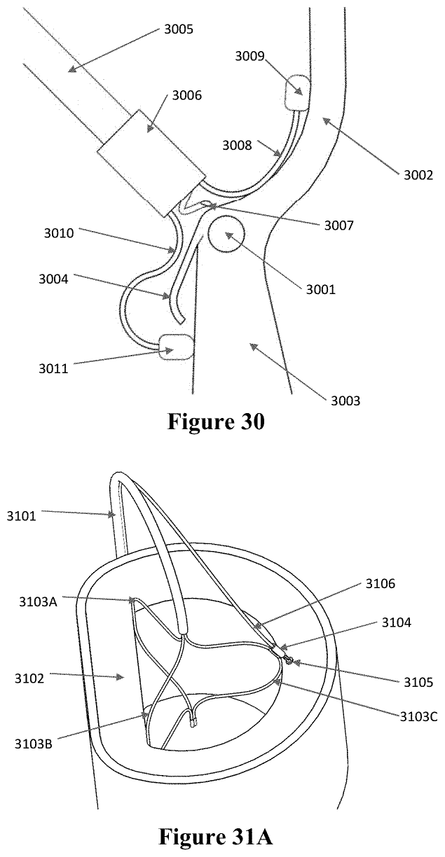

FIG. 30 shows a catheter 3005 for delivering a tissue anchor 3007 to a valve annulus 3001. As illustrated, the valve separates an atrium having an atrial wall 3002 from a ventricle having a ventricular wall 3003, the valve having at least one leaflet 3004. The catheter 3005 as a tip 3006 attached to at least one arm 3008 which interacts with the atrial wall 3002. As shown, the arm 3008 has an atraumatic tip 3009. The catheter tip may also be attached to a second arm 3010 configured to interact with the ventricular wall 3003. As shown, this arm 3010 also has an atraumatic tip 3011. Arm 3010 is configured to leave space around the leaflet 3004 during placement. The configuration of the arms 3008 and 3010 assists in guiding the catheter 3005 to a position allowing the tissue anchor 3007 to attach to the valve annulus 3001.

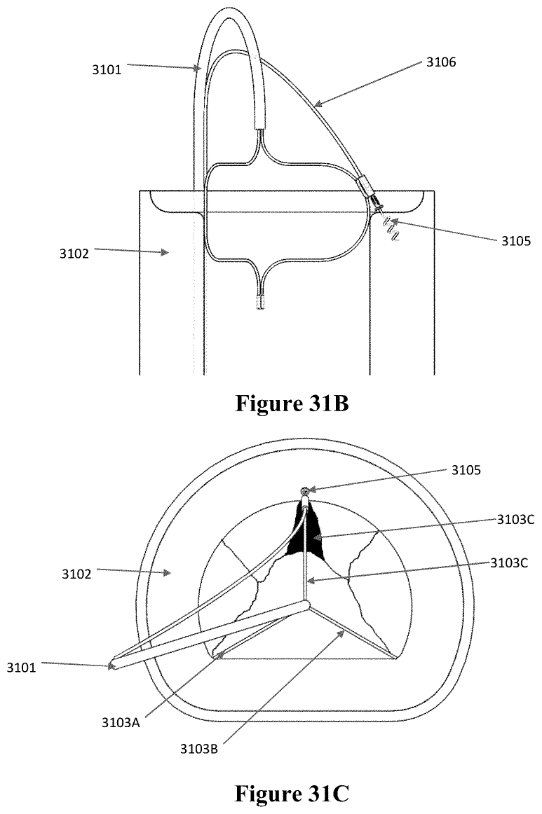

FIG. 31A shows a catheter 3101 having a plurality of arms 3103A-3103C which interact with the annulus of a valve 3102. One arm, 3103C, is slidably attached to an anchor guide 3104, which guides an anchor 3105 toward the annulus of valve 3102. An anchor control wire 3106 is coupled to the anchor 3105, the catheter 3101, and the anchor guide 3104.

FIG. 31B shows the catheter 3101 in place in a valve 3102, the valve being show in section view. In this view, the anchor control wire 3106 and the helical coil of the anchor 3105 are visible.

FIG. 31C shows the catheter 3101 in place in a valve 3102, the catheter 3101 having a plurality of arms 3103A-3103C which interact with the annulus of a valve 3102. One arm, 3103C, compresses a leaflet of the valve 3102 to locate the anchor 3105 relative to the annulus of valve 3102. Arms 3103A and 313B are illustrated as interacting with the valve commissures, having minimal displacement of the valve leaflets.

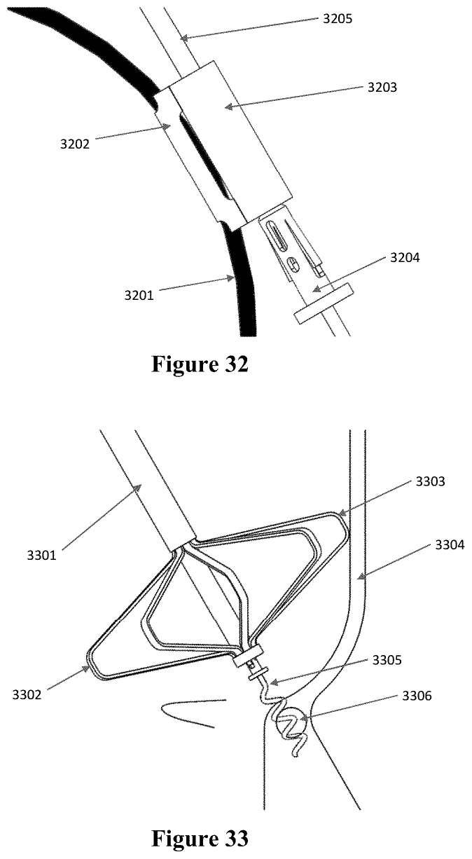

FIG. 32 shows a detail view of an anchor guide 3203 which is coupled to a wire slide 3202 through which a curved wire 3201 passes. An anchor wire 3205 is coupled to the anchor guide 3203 and to the anchor 3204. In the case of a helical anchor, the anchor wire 3205 would be able to rotate within the anchor guide 3203 and would be rotationally fixed to the anchor 3204, allowing the anchor 3204 to screw into the tissue as the anchor wire 3205 is turned remotely, for example, from outside the body.

FIG. 33 shows a catheter 3301 with a plurality of arms 3302. At least one of the arms 3303 interacts with the tissue surrounding the valve area 3304 in order to help direct an anchor 3305 toward a valve annulus 3306. By advancing or retracting the distal end of the arms 3302, the diameter of the structure formed by the arms 3302 can be adjusted to affect anchor 3305 position relative to the valve annulus 3306. The structure is laser cut from tubing or wires made from superelastic or shape memory nitinol, piano or spring stainless steel, shape memory plastic. The shape of the structure can be spherical, oval, tear drop, or other shape deem suitable for the anatomy of the atrium or ventricle where it is deployed. The structure can have arms facing out all around the catheter (i.e., 360 degrees) or partially around the catheter such as but not limited to only on one side of the catheter (i.e., 180 degrees). A pull and push wire or tube can be attached to structure for adjustment of the structure diameter and its removal.

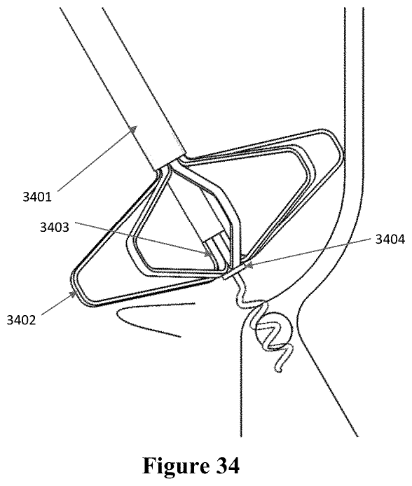

FIG. 34 shows a catheter 3401 with a plurality of arms 3402. The arms 3402 are configured at their distal aspects 3403 to at least partially surround the anchor 3404. In this configuration, a portion of the anchor 3404 resides in the same axial space as the arms 3402, potentially reducing the overall length of the arms 3402 and anchor 3404. The arms 3402 may also be designed to have a delivery configuration with a smaller diameter than the anchor 3404, reducing the diameter of the sheath required for delivery of the catheter 3401 to the valve annulus.

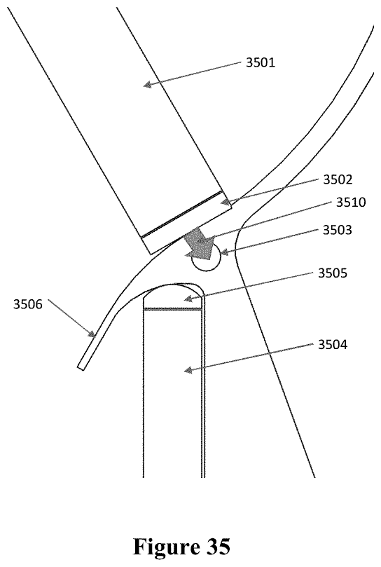

FIG. 35 shows an anchor delivery catheter 3501 having a first magnet 3502 arranged at or near its distal tip, and a target catheter 3504 having a second magnet 3505 arranged at or near its distal tip, the magnets 3502 and 3505 being polarized to attract the two tips of the catheters 3501 and 3504 to each other through the tissue 3506 of the valve in order to direct an anchor 3510 toward a valve annulus 3503. The magnetic material can be made from rare earth samarium cobalt, neodymium, or the like. More than one magnet can be used on each side and can be arranged to orient the location of the anchor with magnetic as well as repulsive attraction. Magnets can be polarized through its thickness as well as diameter or width can be used to direct the anchor to certain location such as away from the leaflet. One of the magnets, either on the target catheter 3504 or second catheter 3501, can be made from a magnetic material such as iron that may or may not be magnetized.

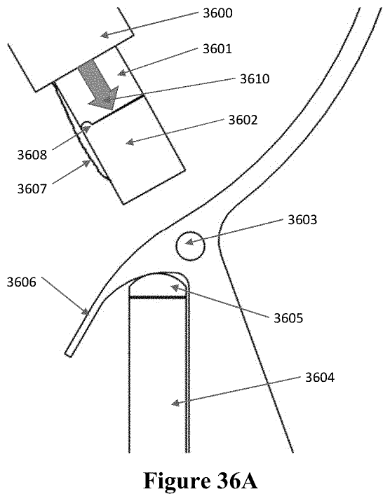

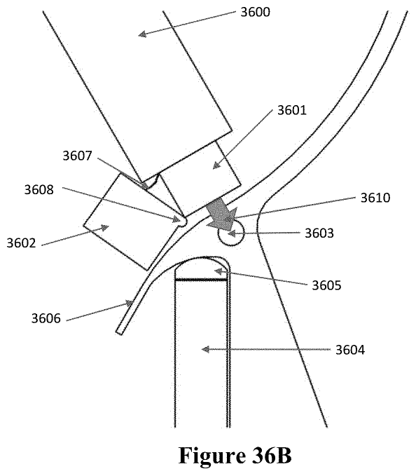

FIG. 36A shows a sheath 3600 through which passes an anchor delivery catheter 3601 having a first magnet 3602 movably arranged at or near its distal tip and a magnet control wire 3607, and a target catheter 3604 having a second magnet 3605 arranged at or near its distal tip, the magnets 3602 and 3605 being polarized to repulse the two tips of the catheters 3601 and 3604 to each other through the tissue 3606 of the valve. Only when the pull wire retracts the magnet 3601 will the surface of the magnet 3608 now facing towards the target catheter attract the magnet 3605 near the tip of the target catheter. The advancement of the catheter tip with the magnet 3601 towards magnet 3605 used to direct an anchor 3610 toward a valve annulus 3603. The anchor delivery catheter 3601 houses an anchor 3610 that is at least partially proximal to the magnet 3602 in the delivery configuration as shown. The magnetic material can be made from rare earth samarium cobalt, neodymium, or the like. More than one magnet can be used on each side and can be arranged to orient the location of the anchor with magnetic as well as repulsive attraction. Magnets can be polarized through its thickness as well as diameter or width can be used to direct the anchor to certain location such as away from the leaflet.

FIG. 36B shows the device of FIG. 36A wherein a tension has been applied to the magnet control wire 3607, pivoting the magnet 3602 to an activated position wherein the magnet 3602 is aside the anchor delivery catheter 3601, and the anchor 3610 can be advanced toward the valve annulus 3603

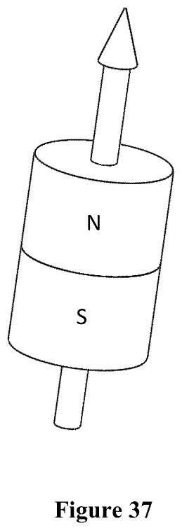

FIG. 37 shows a cylindrical magnet polarized in the axial direction. A magnet in this configuration could be temporarily placed on one side of a valve or valve annulus via a catheter, and arranged so that the desired pole (north or south) is directed across the valve in order to repel like magnetic poles and/or attract opposite magnetic poles on the opposite side of the valve or valve annulus. Such a magnet could also attract un-magnetized ferromagnetic material (for example, iron or nickel compounds) on the opposite side of the valve or valve annulus.

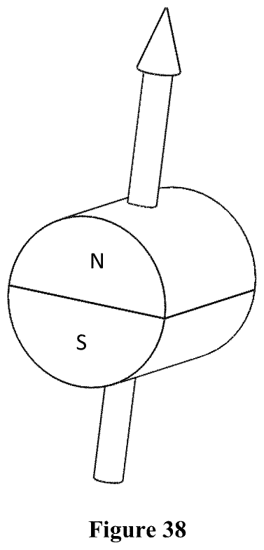

FIG. 38 shows a cylindrical magnet polarized perpendicular to the axial direction. A magnet in this configuration could be temporarily placed on one side of a valve or valve annulus via a catheter and arranged so that an end having both poles is directed across the valve in order to attract un-magnetized ferromagnetic material (for example, iron or nickel compounds) on the opposite side of the valve or valve annulus. Such a magnet would also interact with magnets in certain desirable orientations on the opposite side of the valve or valve annulus.

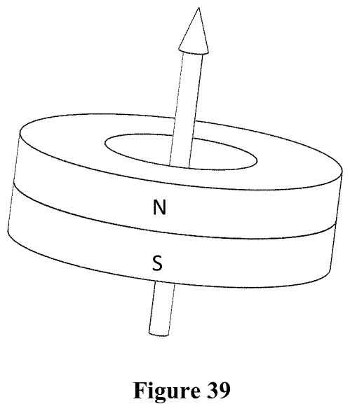

FIG. 39 shows a ring magnet polarized in the axial direction. A magnet in this configuration could be temporarily placed on one side of a valve or valve annulus via a catheter and arranged so that the desired pole (north or south) is directed across the valve in order to repel like magnetic poles and/or attract opposite magnetic poles on the opposite side of the valve or valve annulus. A tissue anchor, guide wire, or other implant or tool could pass through the center of the ring magnet, to affect the desired alignment. Such a magnet could also attract un-magnetized ferromagnetic material (for example, iron or nickel compounds) on the opposite side of the valve or valve annulus.

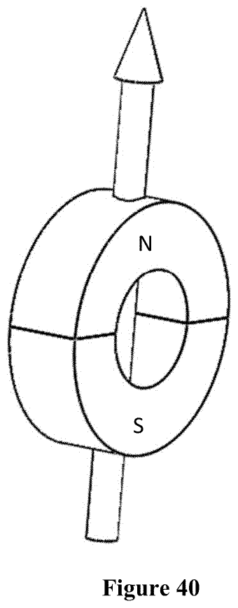

FIG. 40 shows a ring magnet polarized perpendicular to the axial direction. A magnet in this configuration could be temporarily placed on one side of a valve or valve annulus via a catheter and arranged so that an end having both poles is directed across the valve in order to attract un-magnetized ferromagnetic material (for example, iron or nickel compounds) on the opposite side of the valve or valve annulus. Such a magnet would also interact with magnets in certain desirable orientations on the opposite side of the valve or valve annulus.

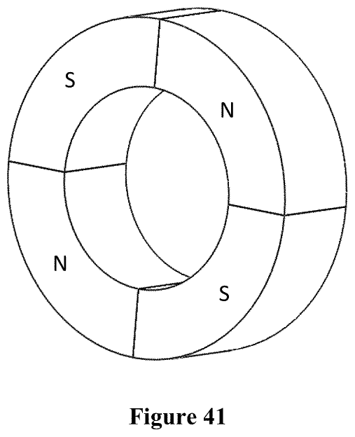

FIG. 41 shows a ring magnet polarized with an even number of poles along its circumference. A magnet in this configuration could be temporarily placed on one side of a valve or valve annulus via a catheter and arranged to interact with a magnet on the opposite side of the valve or valve annulus in order to achieve a rotational and locational alignment. Such a magnet would also interact with magnets in certain desirable orientations on the opposite side of the valve or valve annulus. A tissue anchor, guide wire, or other implant or tool could pass through the center of the ring magnet, to affect the desired alignment.

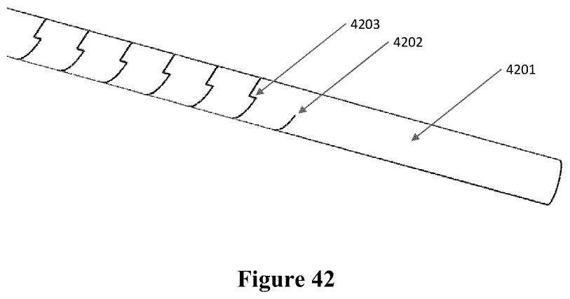

FIG. 42 shows a tube 4201 cut with a spiral 4202 in order to be more flexible than an uncut tube and remain capable of delivering axial forces and torques applied at one end of the tube, for example an end extending out of the body, to the other end of the tube. The spiral 4202 has staggered cuts 4203 that create a self-locking feature, allowing the tube 4201 to transmit more torque than a simple spiral prior to failure.

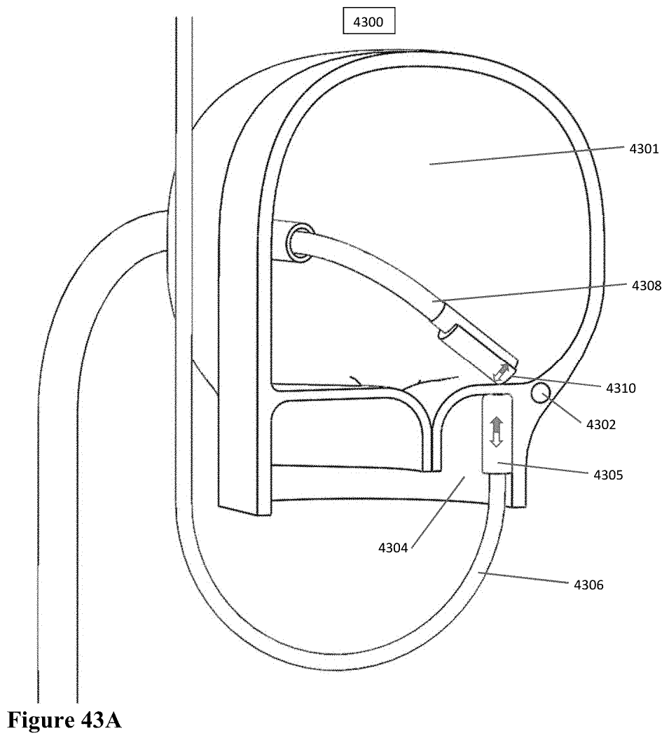

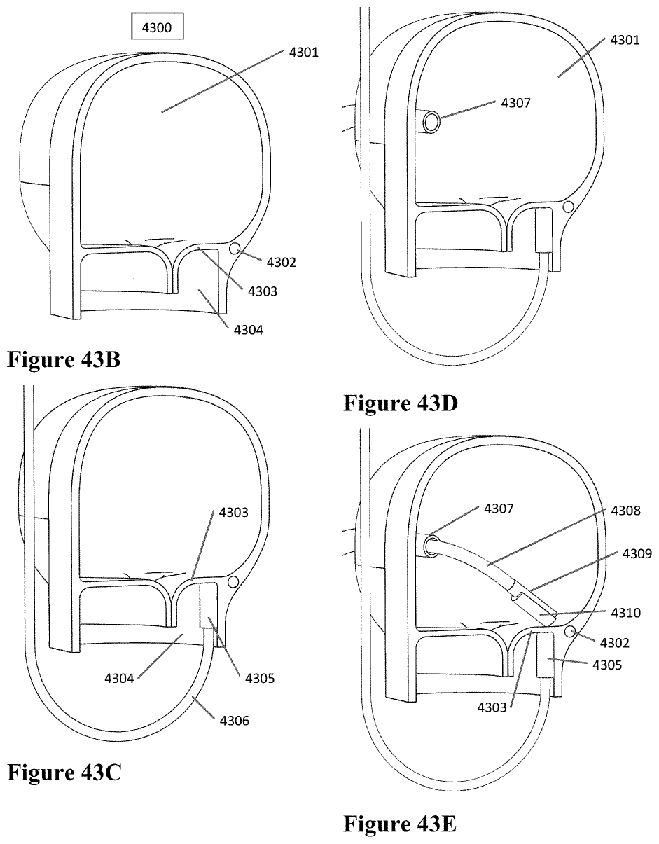

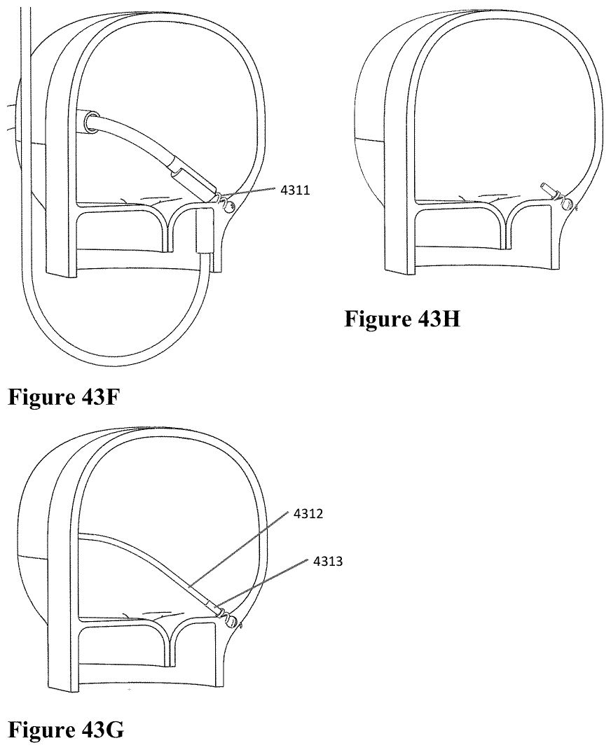

FIG. 43A shows a system for treating a heart 4300 having a target magnet 4305 placed in a ventricle 4304 adjacent a valve annulus 4302, attracting a magnetic tip 4310 of an anchor delivery catheter 4309 in an atrium 4301. The target magnet 4305 is polarized axially such that a first magnetic pole is active on the distal end of the target magnet 4305, while the magnetic tip 4310 of the anchor delivery catheter 4308 is polarized radially, so that the second pole of the magnetic tip 4310 (of opposite polarity to the first magnetic pole at the distal end of the of the target magnet 4305) is pointed toward the target magnet 4305 in the desired position and orientation.

FIG. 43B shows a schematic representation of a heart 4300 having an atrium 4301, a valve annulus 4302, a valve leaflet 4303, and a ventricle 4304

FIG. 43C shows the heart 4300 of FIG. 43B, with a target magnet 4305 attached to a target magnet catheter 4306 placed beneath the leaflet 4303 in the ventricle 4304

FIG. 43D shows the heart of FIG. 43C with a trans-septal access sheath 4307 in place in the atrium 4301.

FIG. 43E shows the heart 4300 of FIG. 43D with an anchor delivery catheter 4308 placed through the trans-septal sheath 4307, the anchor delivery catheter 4308 having a magnetic tip 4310 and an anchor tube 4309. The magnetic tip 4310 is magnetically attracted to the target magnet 4305 through the valve leaflet 4303. The direction and orientation of the distal end of the anchor delivery catheter 4308 can be adjusted by moving the trans-septal sheath 4307 and/or the anchor delivery catheter 4308 so that it points toward the valve annulus 4302.

FIG. 43F shows the heart 4300 of FIG. 43E with a helical tissue anchor 4311 being delivered to the valve annulus.

FIG. 43G shows the heart 4300 of FIG. 43F after delivery of the anchor 4311 and removal of the trans-septal sheath 4307, the anchor delivery catheter 4308, and the target magnet catheter 4306. The anchor 4311 remains attached to an elongate control member 4312. In this configuration, the elongate control member 4312 can be used to direct different aspects of the procedure including placing implants, electrodes, additional anchors, etc.

FIG. 43H shows the heart 4300 of FIG. 43G after removal of the elongate control member 4312. The anchor 4311 remains in the valve annulus. In most situations, the anchor would be coupled to a tissue re-shaping implant (not shown).

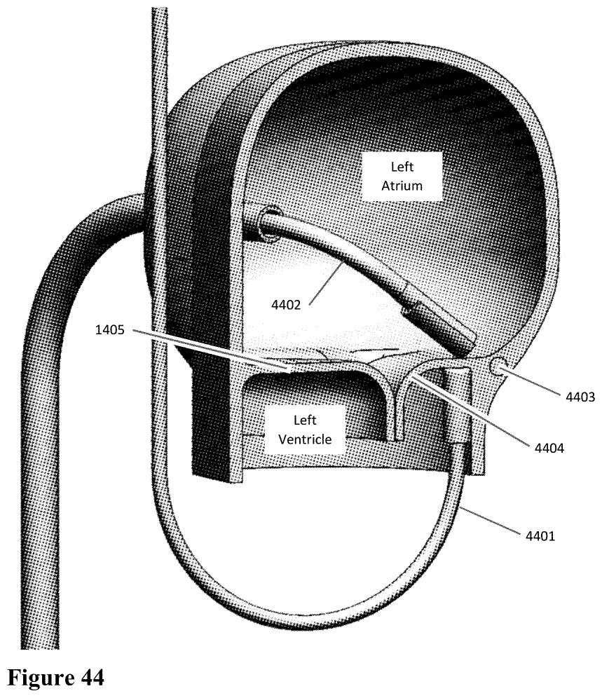

As shown in FIG. 44, a graphical illustration depicts a system of catheters in place in the chambers of a heart. A ventricular catheter 4401 passes through the aorta (not shown) and resides in the left ventricle beneath the mitral leaflets 4404 and 4405, and adjacent the posterior mitral annulus 4403. An atrial catheter 4402 enters the left atrium through the venous system by crossing the septum between the right and left atria. Magnetic attraction aligns the two catheters or positions them adjacent to each other (since they are not in a straight line but can be in an acute angle).

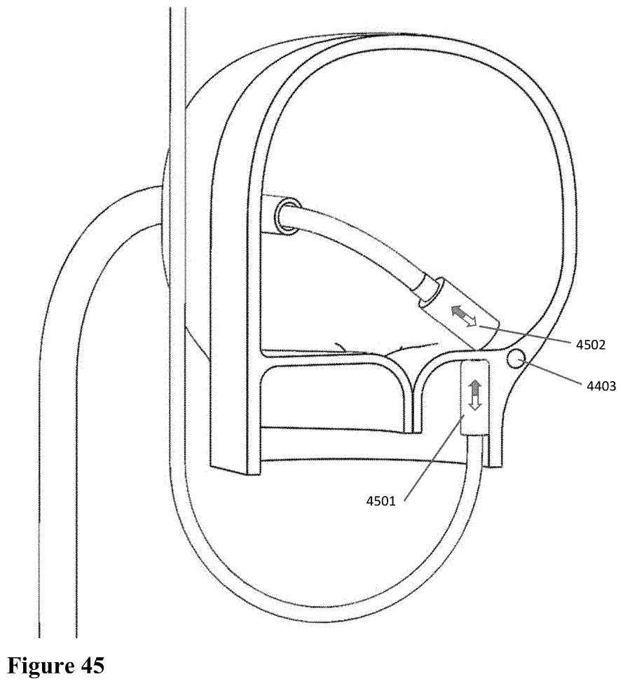

As shown in FIG. 45, a ventricular catheter with target magnet 4501 and an atrial catheter with a locating magnet 4502 reside on opposite sides of a mitral leaflet adjacent a mitral annulus 4403. The target magnet 4501 is polarized axially with a first magnetic pole arranged at the distal end, and the locating magnet 4502 is polarized axially with a second magnetic pole at the distal end. The magnetic poles at the distal ends of each of the magnets 4501 and 4502 are opposite in polarity, causing the magnets to be attracted to each other when the atrial catheter is aimed substantially in the direction of the mitral annulus 4403.

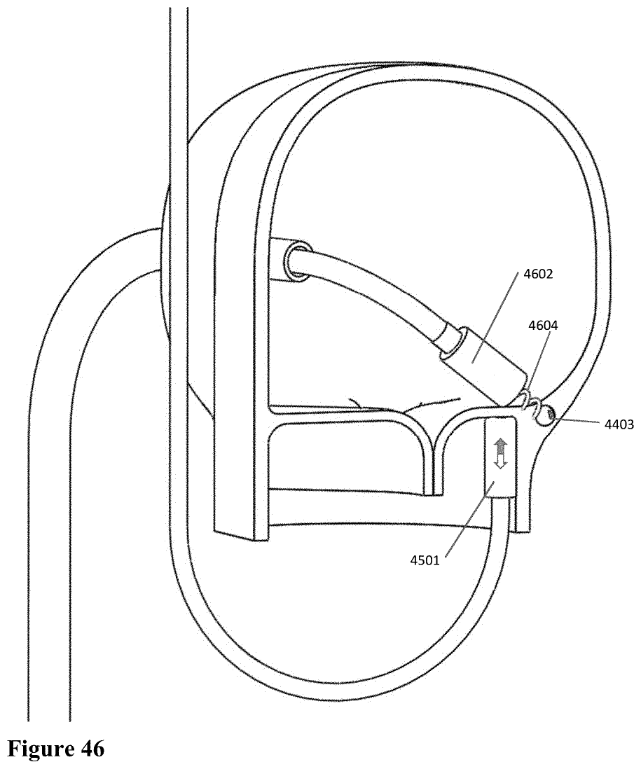

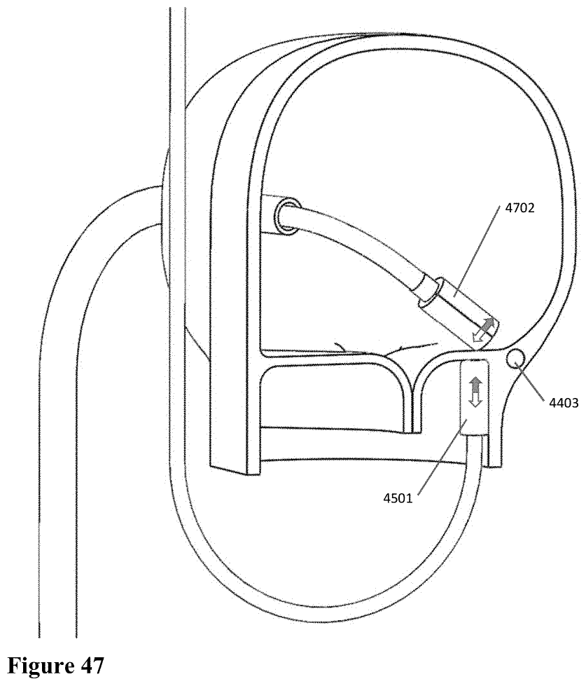

As shown in FIG. 46, a ventricular catheter with target magnet 4501and an atrial catheter with a locating tip 4602 constructed of a non-magnetized ferro-magnetic material reside on opposite sides of a mitral leaflet adjacent a mitral annulus 4403. The target magnet 4501 is polarized axially, and the locating tip 4602 is not magnetized. This arrangement causes the target magnet 4501 and the locating tip 4602 to be attracted to each other regardless of their precise orientation. A helical tissue anchor 4604 extends from the distal end of the atrial catheter and can be directed in the direction of the mitral annulus 4403.

As shown in FIG. 47, a ventricular catheter with target magnet 4501 and an atrial catheter with a locating magnet 4702 reside on opposite sides of a mitral leaflet adjacent a mitral annulus 4403. The target magnet 4501 is polarized axially with a first magnetic pole arranged at the distal end, and the locating magnet 4702 is polarized radially with a second magnetic pole on one side. The locating magnet 4702 substantially surrounds the lumen of the atrial catheter. The magnetic poles of each of the magnets 4501 and 4702 are opposite in polarity, causing the magnets to be attracted to each other when the atrial catheter is rotated to so the second pole faces the target magnet 4501, and so that it is aimed substantially in the direction of the mitral annulus 4403.

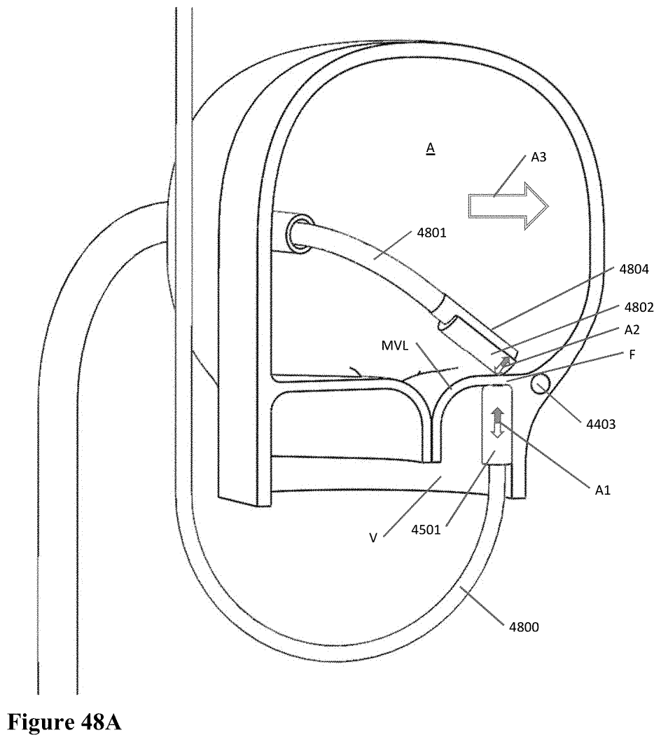

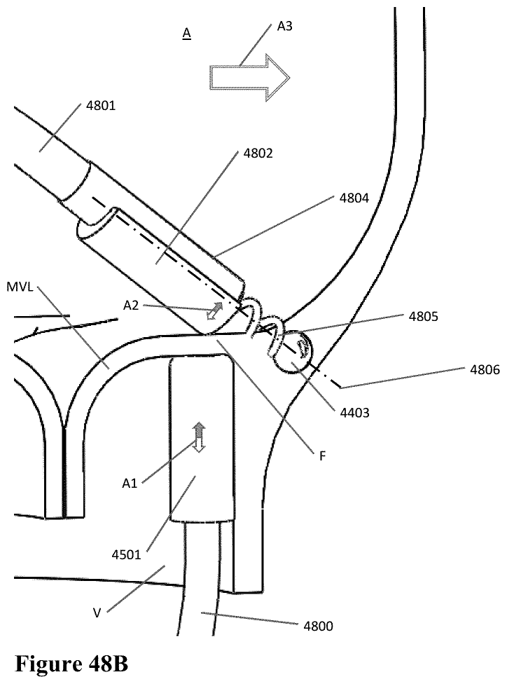

As shown in FIG. 48A, a locating catheter 4800 configured for advancement into the patient's ventricle V has a target magnet 4501 at its distal end. An anchor delivery catheter 4801 configured for advancement into the patient's atrium A has a magnet 4802 at its distal end. The target magnet 4501 and the positioning magnet 4802 are configured to reside on opposite sides of a mitral leaflet MVL adjacent a mitral annulus 4403. The target magnet 4501 is polarized axially as indicated by arrow Al with a pole having a first magnetic polarity oriented toward the distal end. The positioning magnet 4802 is polarized radially as indicated by arrow A2 with a pole having a second magnetic polarity along one side. The first and second magnetic polarities are opposite to each other so that the distal ends of the catheters attract and self-orient as shown in FIG. 48B. The magnetic attraction creates a virtual fulcrum at F a location near the attachment base of the leaflet MVL to the annulus 4403 which allows pushing and pulling (axially advancing and retracting) on the catheter shaft to cause the distal end of the anchor delivery catheter to pivot about the fulcrum and aim a delivery path 4806 at a target region in the annulus 4403. The locating catheter 4800 may optionally incorporate an anchor guide 4804 that maintains the alignment between the helical or other anchor 4805 and the positioning magnet 4802.

The positioning magnet 4802 has a “half-cylindrical” design (e.g. subtending an arc from 120° to 210°) which partially surrounds an anchor delivery lumen of the anchor delivery catheter 4801. Such a design may be advantageous compared to a hollow cylindrical magnet when directing a helical or other tissue anchor 4805 relative to the target magnet 4501 by radially offset the anchor delivery lumen within the guide catheter. Thus, when the anchor delivery catheter 4801 is rotated about its longitudinal axis to align the first pole with the second pole on the positioning magnet 4501, the offset facilitates aiming the delivery path toward the annulus target 4403. The design may also reduce the risk of damage to the positioning magnet 4502 during manufacture, shipping, or use. A thicker partial thickness of a half-cylindrical design may provide a stronger magnetic field than available with a thinner full cylindrical (tubular) magnet having the same outer diameter.

As shown in FIG. 48B, the catheter system of FIG. 48A is used to deliver a helical or other anchor 4805 to a mitral annulus 4403. The anchor 4805 may be deployed by axial advancement along the delivery path 4806 from the anchor guide 4804. A preferred delivery path 4806 extends laterally outwardly from the fulcrum F where the distal end of the anchor delivery catheter 4801 is magnetically coupled to the distal end of the locating catheter 4800. As shown, the annulus 4403 is disposed radially outwardly (in a direction A3 away from the septum or the root of the aorta) relative to the anchor delivery catheter magnet 4501. This arrangement may be advantageous when the target magnet 4501 resides in a body cavity (the left ventricle as shown) that does not contain the target tissue, in this case the mitral annulus 4403.

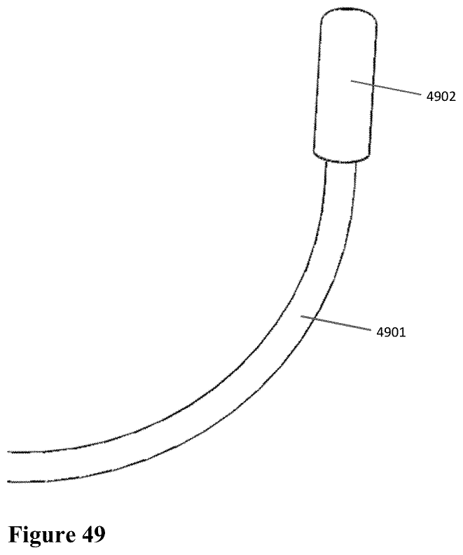

As shown in FIG. 49, a catheter 4901 has a magnetic tip 4902 that completely closes the distal end of the catheter 4901. The magnetic tip may be polarized axially, radially, at an angle to the axis, or with a more complex pattern of polarization zones. For example, this catheter may be inserted into a chamber of a heart to act as an attractive target catheter for a second catheter in an adjacent chamber.

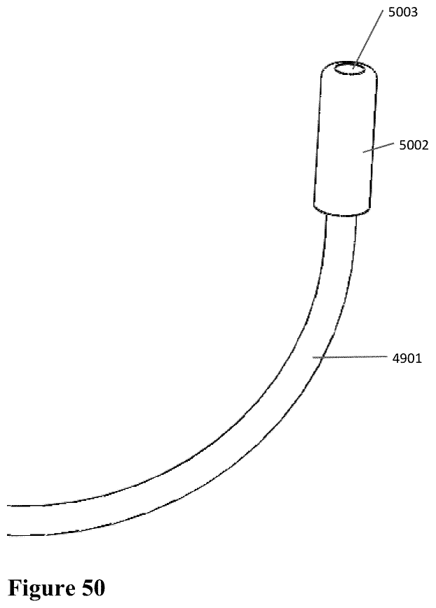

As shown in FIG. 50, a catheter 4901 has a magnetic tip 5002 coupled to the distal end of the catheter 4901. A lumen 5003 extends substantially through the magnetic tip 5002 and communicates with an inner lumen of the catheter 4901. The lumen 5003 may allow passage of a guidewire which may assist in placement of the catheter 4901 and magnetic tip 5002 in a desired location in the heart or other advantageous anatomy. The catheter 4901 has a curvature as shown that may be advantageous in accessing certain target anatomy. The curvature may be formed into the resilient material of the catheter 4901, may be actively adjusted by means of inner tension and compression members within the catheter 4901, or may be bent to shape in situ by other means (not shown.) An outer tube with one or more lumen (not shown) can also extend from the proximal end of the magnet adjacent to the catheter tubular body.

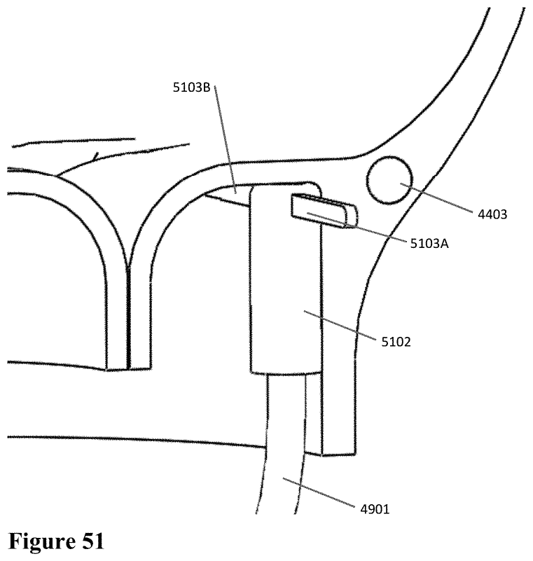

As shown in FIG. 51, a catheter 4901 delivers a magnetic tip 5102 to a target position in a heart chamber adjacent an annulus 4403. Arms 5103A and 5103B extend outward from the magnetic tip 5102 in substantially opposite directions, to form an image reference aligned relative to the valve annulus 4403, to assist in orienting and placing a template in an adjacent chamber of the heart. Arms 5103A and 5103B are constructed to be visible under cardiac imaging technology such as ultrasound or fluoroscopy, by including in the construction of arms 5103A and 5103B radiopaque material, echogenic material, or a combination of one or more materials.

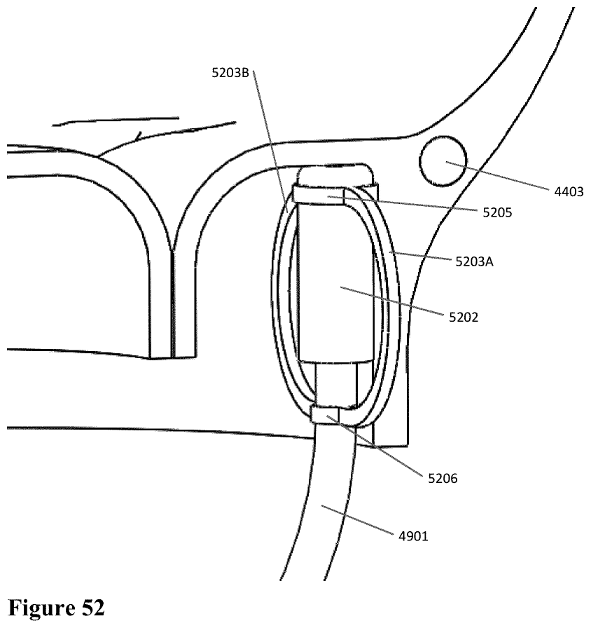

As shown in FIG. 52, a catheter 4901 delivers a magnetic tip 5202 to a target position in a heart chamber adjacent an annulus 4403. Bent arms 5203A and 5203B extend outward from the magnetic tip 5202 in substantially opposite directions, to form an image reference aligned relative to the valve annulus 4403, to assist in orienting and placing a template in an adjacent chamber of the heart. Bent arms 5203A and 5203B are constructed to be visible under cardiac imaging technology such as ultrasound or fluoroscopy, by including in the construction of arms 5203A and 5203B radiopaque material, echogenic material, or a combination of one or more materials. Bent arms 5203A and 5203B are connected at the distal end by a fixed ring 5205, and at the proximal end by a movable ring 5206 such that the bent arms 5203A and 5203B can be extended from the magnetic tip 5202 to provide a visual reference for orientation, or retracted against the magnetic tip 5202 for access to and removal from the target site in the anatomy.

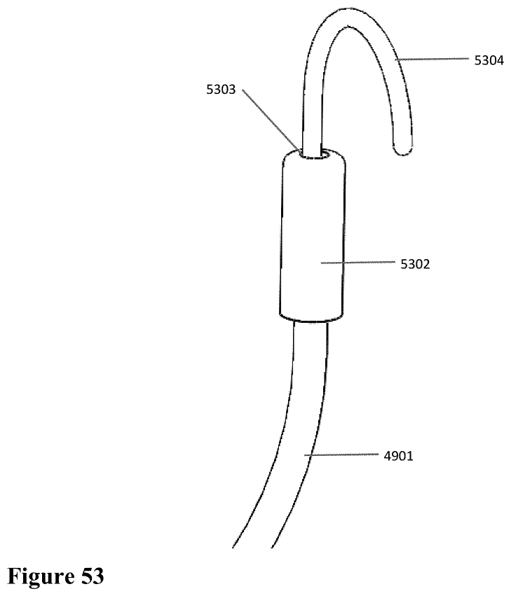

As shown in FIG. 53, a catheter 4901 has a magnetic tip 5302 coupled to the distal end of the catheter 4901. A lumen 5303 extends substantially through the magnetic tip 5302 and communicates with an inner lumen of the catheter 4901 or the lumen of an outer tube (not shown). The lumen 5303 may allow passage of one or more guidewires 5304 which may guide the catheter through the vasculatures or valves and assist in placement of the catheter 4901 and magnetic tip 5302 in a desired location in the heart or other advantageous anatomy. One or more guidewires 5304 (one shown) may be constructed with echogenic materials, radiopaque materials, structural materials or a combination of one or more such materials, so that the curve of the guidewire 5304 can present a visual guide to orientation of an implant placed relative to the curve of the guidewire 5304. When more than one guidewires is used, they can be coupled such that the angle tips curve in substantially opposed directions. A single guidewire with a tip consisting of multiple filaments biased to bend in substantially opposed directions offer similar utility.

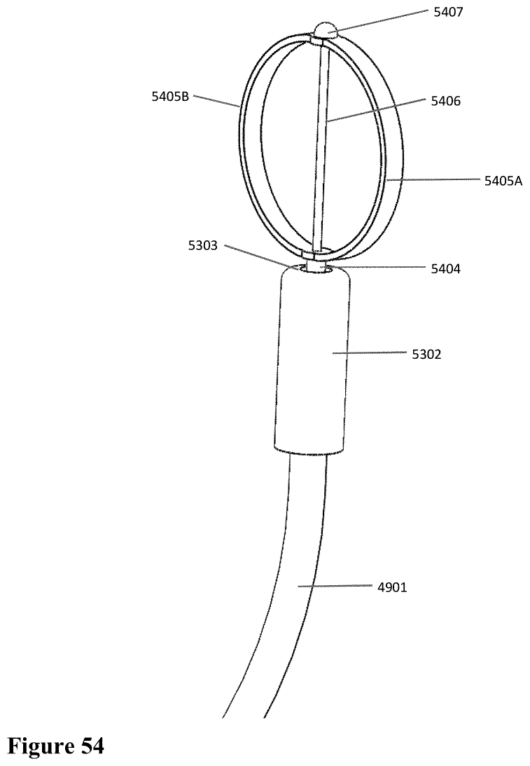

As shown in FIG. 54, a catheter 4901 has a magnetic tip 5302 coupled to the distal end of the catheter 4901. A lumen 5303 extends substantially through the magnetic tip 5302 and communicates with an inner lumen of the catheter 4901 or the lumen of an outer tube (not shown). The lumen 5303 may allow passage of a guidewire 5404 which may assist in placement of the catheter 4901 and magnetic tip 5302 in a desired location in the heart or other advantageous anatomy. An indicating device 5404 may be constructed with echogenic materials, radiopaque materials, structural materials or a combination of one or more such materials, so that the bent arms 5405A and 5405B of the indicating device 5404 can present a visual guide to orientation of an implant placed relative to the curve of the indicating device 5404. Bent arms 5405A and 5405B are connected at the distal end to a movable wire 5407, and at the proximal end to a stationary lumen 5404 such that the bent arms 5405A and 5405B can be extended outwardly to provide a visual reference for orientation or retracted inwardly for passage through the catheter 4901.

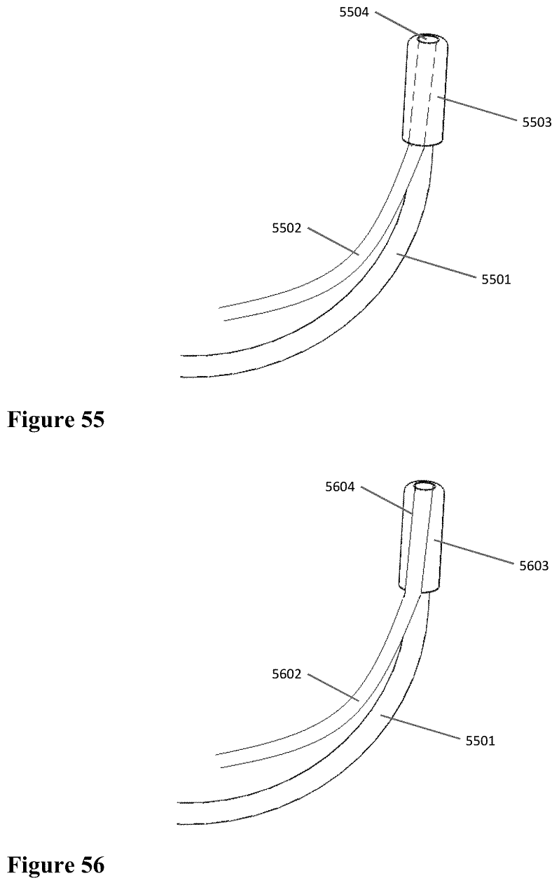

As shown in FIG. 55, a catheter has a primary shaft 5501, an accessory lumen 5502 and a magnetic tip 5503. The magnetic tip 5503 has as lumen 5504 extending substantially through it and communicating with the accessory lumen 5502. The magnetic tip 5503 is coupled to the primary shaft 5501. The primary shaft 5501 may be curved to fit the target anatomy, steerable to fit the target anatomy, or flexible to allow access through vasculature and heart valves to reach the target anatomy.

As shown in FIG. 56, a catheter has a primary shaft 5501, an accessory lumen 5602 and a magnetic tip 5603. The magnetic tip 5603 has as partial lumen 5604 extending through at least a portion of the magnetic tip 5603 and communicating with the accessory lumen 5602. The magnetic tip 5603 is coupled to the primary shaft 5501. The primary shaft 5501 may be curved to fit the target anatomy, steerable to fit the target anatomy, or flexible to allow access through vasculature and heart valves to reach the target anatomy.

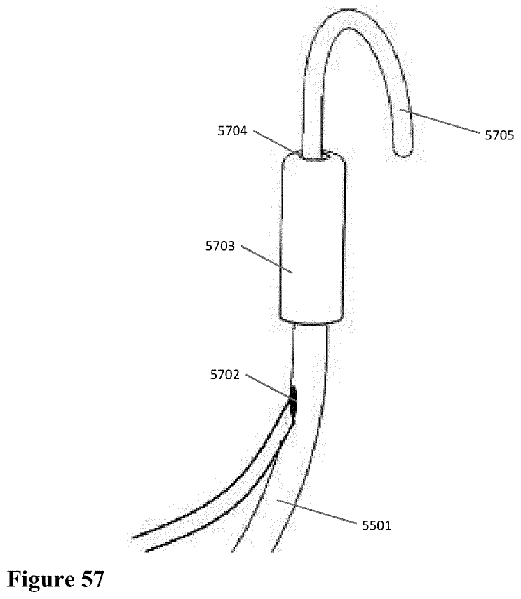

As shown in FIG. 57, a catheter has a primary shaft 5501 having a side port 5702 and a magnetic tip 5703. The magnetic tip 5703 has as lumen 5704 extending through at least a portion of the magnetic tip 5703 and communicating with the side port 5702. The magnetic tip 5703 is coupled to the primary shaft 5501. The primary shaft 5501 may be curved to fit the target anatomy, steerable to fit the target anatomy, or flexible to allow access through vasculature and heart valves to reach the target anatomy. A guidewire 5705 is placed through the side port 5702 and the lumen 5704. The guidewire 5705 may help with placement of the catheter, as well as providing an imaging reference for alignment of an implant placed in a nearby anatomic location.

### EXAMPLES

In one example, an elongate device is attached at the distal end to one or more locating elements. In a further example, the locating elements contact tissues and position the distal end of the elongate device relative to those tissues. In a further example, the locating elements are radiopaque, making them visible on fluoroscopic examination. In another example, the locating elements include echogenic features. In a further example, the echogenic features are retro-reflective surface textures. In another example, the echogenic features are surface textures that scatter sound waves. In another example, the echogenic features are materials of different densities within the locating elements. In a further example, the echogenic materials are hollow pores contained in the material of the locating element. In another example, the echogenic materials are hollow beads contained in the material of the locating element. In another example, the locating elements are constructed of layers of materials having different densities.

In one example, a tissue anchor delivery catheter has at tip with a small diameter configuration for passage through an introducer sheath, and a second configuration with extended tissue locating arms. In a further example, these tissue locating arms have different shapes for interacting with tissue on opposed sides of a valve or valve annulus. In a further example, the tissue arms have atraumatic tips which may be formed directly on the ends of the arms, or made separately and attached to the distal ends of the arms. In a further example, one or more of the arms has a configuration that allows it to go around other valve structures, including valve leaflets, and press on alternate heart structures including the wall of the atrium, the wall of the ventricle, the valve annulus, or other nearby structures. In a further example, the catheter tip has an internal lumen capable of passing a tissue anchoring mechanism.

In one example, a tissue anchor delivery catheter has a 2 or more locating wires that deflect to locate at least a portion of the catheter relative to at least a portion of the target anatomy. In a preferred example, the delivery catheter has 3 locating wires. In a further example, at least one anchor guide is disposed at least partially around at least one of the locating wires and slidable relative to the locating wire. In a further example, the locating wire is curved in the area engaged with the anchor guide. In a further example, the anchor guide has spaces to allow passage of a curved wire in one direction, such that the curve of the wire urges the anchor guide to a desired rotational alignment relative to the locating wire and target anatomy. In a further example, the tissue anchor is engaged with the anchor guide via a flexible anchor control wire. In a further example, the anchor control wire transmits a torque applied at the proximal end to the anchor disposed at the distal end.

In one example, the 6.5 Fr steerable catheter with a tear drop shaped superelastic nitinol basket structure on the outside and the anchor in its lumen is folded and inserted into a 13.8 Fr introducer sheath which has transseptally crossed the atrial wall. Upon exit of the sheath, the basket is opened to a tear drop shape that has curves that are similar to the atrial wall just superior to the atrial annulus. The catheter tip is directed toward the annulus while the edge of the basket is intermittently in contact with the posterior atrial wall. When the catheter tip reaches the annulus and is stabilized by the basket against the wall, the basket size is adjusted with a pull wire and the catheter is deflected such that the tip of the catheter is aligned with the annulus. The anchor is then deployed into the annulus at a distance from the atrial wall determined by the radius of the basket. The catheter with its basket is removed from the 13.8 Fr catheter. A tissue shaping template, or other implant, can then be attached to the anchor.

In another example, a steerable 8 Fr catheter, the target catheter, with a magnet tip is inserted into the femoral artery and advanced retrograde through the aorta. The tip of the catheter is directed towards to posterior wall of the left ventricle and up beneath the posterior mitral leaflet. It is then wedged against the annulus on the ventricle side and held in place adjacent to P2 mitral leaflet. A steerable catheter with a locating magnetic tip such as that displayed in FIG. 36 is then inserted through a transseptal access sheath placed transseptally across the atrial wall and into the left atrium. The locating magnetic tip is then directed in the annulus adjacent to the P2 leaflet. A pull string is attached to the magnet to expose the opposite end of the magnet which has an attraction towards the distal end of the target magnet catheter. When the magnetic fields of the two magnets, having opposite poles, are attracted to each other with the annulus tissues in between, they stick together and hold the catheter tip in place with minimal slippage and stabilize the catheter position against motion caused by the contraction of the heart. The anchor can then be deployed into the annulus adjacent to the magnet. After anchoring, the locating catheter is removed from the transseptal access sheath. A tissue shaping template, or other implant, can then be attached to the anchor.

In one example, an anchor control wire is formed of a tube with laser cut features to allow increased flexibility in a specific flexible segment of the tube. In a further example, the laser cut features include a spiral cut. In a further example, the spiral cut includes staggered cut sections that interlock when a torque is applied to at least one end of the control wire. In a further example, the laser cut features are at an angle to the axis of the tube. In an additional example, the laser cut features are formed along a line which does not intersect the axis of the tube.

In one example, an elongate device is attached at the distal end to one or more probe elements. In a further example, the probe elements deflect in contact with bodily tissues. In a further example, the probe elements are radiopaque, making them visible on fluoroscopic examination. In another example, the probe elements include echogenic features. In a further example, the echogenic features are retro-reflective surface textures. In another example, the echogenic features are surface textures that scatter sound waves. In another example, the echogenic features are materials of different densities within the probe elements. In a further example, the echogenic materials are hollow pores contained in the material of the probe element. In another example, the echogenic materials are hollow beads contained in the material of the probe element. In another example, the probe elements are constructed of layers of materials having different densities.

In one example, the probe elements are configured to fold inward to a reduced profile, enabling the elongate device with probe elements to pass through a smaller lumen than when the probe elements are extended. In a further example, the elements fold distally and inward. In another example, the elements fold proximally and inward.

In one example, the elongate device includes an instrument channel for delivering a device to treat, anchor to, mark, or otherwise affect the target tissue. In another example, the instrument channel is centered in the elongate device. In another example, the elongate device contains more than one instrument channel.

In one example, the probe elements are configured to deform as the elongate device tip approaches a section of target tissue. In a further example, this deformation will be visible via one or more imaging modalities (that is, ultrasonography, fluoroscopy, CT scan, MRI, etc.) In a further example, the probe elements flex in response to tissue movement, giving an indication of tissue motion visible on one or more imaging modalities.

In one example, all of the probe elements are substantially the same length. In another example, the elongate device includes probe elements having two or more different lengths. In a further example, one or more probe elements has a long length, and one or more probe elements has a short length. In a further example, probe elements have three or more distinct lengths. In another example, two or more probe elements each have a distinct length. In another example, the elongate device can be rotated relative to the target tissue to bring probe elements of the desired configuration into alignment with the target tissue to refine positioning.

In one example, the probe elements have a substantially consistent cross section along their length. In a further example, the probe elements have one or more sections of reduced cross section. In another example, the probe elements have a cross section that varies along the length of the element.

In one example, one or more probe elements form a single band from the elongated device to the distal end of the element. In another example, one or more probe elements have one or more branches extending off the side creating a second distal or proximal endpoint. In another example, one or more probe elements have one or more branches extending from the end of the probe element. In another example, one or more probe elements have branches extending from the side or end of the probe element and connecting to one or more adjacent probe elements. In another example, two adjacent probe elements are connected at the distal end to allow greater contact with the tissue without substantial increase in mass. In a further example, two adjacent probe elements are connected at the distal end by a foldable branch, which allows the distal ends of the adjacent probe elements to move closer to each other so that they can be delivered through a smaller diameter than in the extended configuration.

In one example, one or more probe elements bend near the junction with the elongated device, and continue in a substantially straight direction to the distal end of the element. In another example, one or more probe elements have a bend disposed at some distance from the junction with the elongated device. In a preferred example, one or more probe elements have a first bend near the junction of the elongated device, and a second bend in substantially the same direction distal to the first bend. In another example, one or more probe elements have a first bend near the junction of the elongated device, and a second bend in substantially the opposite direction distal to the first bend. In another example, one or more probe elements have a continuous bend along a substantial portion of their length.

In one example, one or more probe elements branch to create a probe segment that bends near the branching point. In a preferred example, the probe segment extends inward and distally from the branching point. In another example, the probe segment extends inward and proximally from the branching point. In another example, the probe segment extends outward and distally from the branching point. In another example, the probe segment extends outward and proximally from the branching point.

In one example, each element is coupled to an elongate structure so that the element can be moved or manipulated into position or to a different position. In a further example, the elongate structure comprises suture, wire or the like. In another example each probe element is independently movable.

In one example, one or more probe elements are attached to an elongate structure which is remotely actuated. In another example, two or more elongate structures are independently remotely actuated. In a further example, one or more probe elements include a sensor which can be read remotely.

In one example, an elongate device having at least one hollow channel is attached at the distal end to one or more probe elements. In a further example, the elongate device is a sheath. In a further example, the sheath includes a hemostatic valve. In a further example, the sheath can be steered by controls located outside the body.

In one example, an elongate device having at least one hollow channel is attached at the distal end to one or more probe elements, and an expandable structure is contained within the hollow channel. In a further example, the expandable structure is pushed distally to release it from the elongate device. In a further example, the expandable structure self-expands upon release from the elongate structure. In another example, the expandable structure is a stent. In another example, the expandable structure contains an artificial valve.

In one example, an elongate device is attached to one or more probe elements at the distal end, the elongate device being placed at least partially through an outer elongate device. In a further example, the outer elongate device is a sheath. In a further example, the outer elongate device contains an expandable structure. In a further example, the elongate device with probe elements is disposed at least partially within the expandable structure. In another example, the elongate device with probe elements is disposed alongside the expandable structure.

In one example, an elongate device is attached to one or more probe elements at the distal end, the elongate device being placed at least partially through sheath. In a further example, the sheath contains an implant. In a further example, the elongate device with probe elements is disposed at least partially within the implant. In another example, the elongate device with probe elements is disposed alongside the implant.

In one example, an implant is attached to probe elements. In a further example, the implant has a delivery configuration and an implanted configuration. In a further example, the probe elements deflect to interact with tissue when the implant is in the deployment configuration. In a further example, the probe elements are held against the tissue when the implant is in the implanted configuration. In one example, an elongate device having an instrument channel is attached to one or more probe elements at its distal end, and a tissue coupling anchor is contained at least partially within the instrument channel. In a further example, the elongate device is placed in apposition with target tissue using, while the probe elements aid in visualizing the positional relationship between the target tissue and the elongate device. In a further example, the tissue coupling anchor consists of an implant portion and a delivery portion. In a further example, retracting the tissue coupling anchor proximally brings it proximal to the probe elements. In a further example, retracting the tissue coupling anchor proximally positions the distal end of the tissue coupling anchor within the instrument channel, and extending the tissue coupling anchor distally places the distal tip of the tissue coupling anchor into apposition with the target tissue. In a further example, turning the delivery portion turns the implant portion causing it to helically penetrate the target tissue. In a further example, the delivery portion of the tissue coupling anchor can be detached from the implant portion.

In one example, an elongate device has probe elements attached to its distal end and at least partially contains a tissue shaping template, the elongate device and tissue shaping template are slidably disposed around a tissue coupling anchor which is coupled to the target tissue. In a further example, the elongate device and tissue shaping template are advanced distally over the tissue coupling anchor until the probe elements deflect in contact with the target tissue. In a further example, the elongate device containing the tissue shaping template is rotated about the tissue coupling anchor to align the tissue shaping template with the target tissue. In a further example, the tissue shaping template is coupled to the tissue coupling anchor and released from the elongate device. In a further example, the elongate device is rotated, advanced and/or retracted so that the probe elements contact the tissue shaped by the tissue shaping template, aiding in visualization of the shaped tissue, and verification of the desired tissue shaping effect.

In one example, an elongate device has an array of probe elements disposed along at least a portion of its length, and the elongate device is placed adjacent to target tissue. In a further example, a first feature of the target tissue deflects a first region of probe elements on the elongate device, indicating the location of this first feature of the target tissue. In a further example, a second feature of the target tissue deflects a second region of probe elements on the elongate device, indicating the location of this second feature of the target tissue as well as the distance between the first feature and the second feature. In a further example, at least one feature of the target tissue is a valve.

In one example, an elongate device has one or more probe elements coupled to its distal end, the elongate device having a cross section which has one lumen, more than one lumen, or no lumens. In a further example, the cross section having no lumens has a shape that is triangular, quadrilateral, pentagonal, hexagonal, heptagonal, octagonal, nonagonal, decagonal, or a polygon having a greater number of sides. In another example, the cross section having no lumens is circular, oval, elliptical, or another predominantly round shape. In another example, the cross section having no lumens has an arcuate shape, and “L” shape, a “C” shape, or comprises a partially open channel.

In one example, an elongate device has one or more probe elements coupled to its distal end and is comprised of two or more elongate sections connected to each other by angled rungs. In a further example, changing the relative position of the elongate sections in a proximal-distal direction changes the height, or width, or diameter of the elongate section.

In one example, an elongate device is coupled to a stationary hub, which is coupled to at least one end of one or more probe elements, another end of at least one of the probe elements being coupled to a movable hub slidably engaged with the elongate device. In a further example, one or more probe elements bend outward, away from the elongate device to form a bulge. In a further example, moving the movable hub towards the stationary hub increases the diameter of the bulge, and moving the movable hub away from the stationary hub decreases the diameter of the bulge. In a further example, the adjustable diameter of the bulge is used to visualize the diameter of a body structure.

In one example, an elongate device is attached to at least one probe element at or near the distal end of the elongate device, the at least one probe element comprising a first material and a second material. In a further example, the difference in properties between the two materials enhances imaging of the probe element. In another example, the different electrical properties between the two materials send information about the conditions in the region of the probe to a display located outside the body. In a further example, the information includes one or more of the following: strain in the probe element, pressure, temperature, electrical conductivity, oxygen saturation. In another example, the second material itself comprises a sensing device capable of sending information to a display located outside the body. In a further example, the information the sensing device sends to the display includes one or more of the following: strain in the probe element, pressure, temperature, electrical conductivity, oxygen saturation. In another example, one of the materials of the probe element is electrically conductive and communicates electrical information such as EKG measurements to a display located outside the body.

In one example, an elongate device is attached to at least one probe element, and an adjustment member is slidably coupled to the elongate device and contacts tone or more probe elements. In a further example, adjusting the proximal to distal position of the adjustment member relative to the probe elements changes the effective length of the probe element. In a further example, moving the adjustment member distally relative to the probe elements makes the effective length of the probe element shorter, and moving the adjustment member proximally relative to the probe elements make the effective length of the probe element longer. In a further example, the effective length of the probe elements is adjusted to a relatively long position during initial positioning of the elongate member relative to the target tissue and adjusted to a relatively shorter position during final positioning, allowing for variable positional precision as needed.

In one example, a first elongate device having one or more probe elements having a first length coupled to its distal end is slidably coupled to a second elongate device having one or more probe elements having a second length coupled to its distal end. In a further example, the probe elements of the first elongate device can be extended distal to the probe elements of the second elongate device or can be retracted proximally to the probe elements of the second elongate device. In a further example, the probe elements of the first elongate device are longer than the probe elements of the second elongate device. In a further example, the first elongate device is arranged to be the distalmost for initial positioning of the coupled elongate devices relative to the target tissue, then the second elongate device is arranged to be the distalmost for precise final positioning of the coupled elongated devices. In a further example, a tissue coupling anchor is slidably coupled to the coupled elongate members and configured to couple to the target tissue once final positioning has been achieved.

In one example, and elongate device contains two or more independently positionable arms having probe elements disposed along their length. In a further example, the arms having probe elements are used to locate a linear structure in the target tissue. In another example, an elongate device contains three or more independently positionable arms having probe elements disposed along their length. In a further example, the three arms having probe elements are used to locate a planar structure in the target tissue. In a further example, the planar structure is a heart valve. In another example, the elongate device is attached to one or more probe elements, and acts as one of the independently controllable arms.

In another example, a device is attached to probe elements. In a still further example, the device is a therapeutic device. In another example, the device is a diagnostic device. In yet another example, the device is a locating or positioning device. In a further example, the device is a sheath with a channel capable of delivering at least one therapeutic, diagnostic, positioning, locating, or marking device.

In a preferred example, the target magnet is coupled to a target catheter for delivery adjacent the target tissue. In a further example, the target magnet includes features that align with the target tissues. In a further example, these aligning features are visible under fluoroscopy, ultrasound imaging, or a combination of the two. In a further example, the alignment features are coupled directly to the target magnet. In a further example, the alignment features are configured to be extensible outward from and inward towards the target magnet. In a preferred example, the alignment features have a structural component comprising superelastic nitinol, and an echogenic component comprising ePTFE, or knitted/woven polyester. In another example, the alignment features are coupled to a wire passed through a lumen of the target catheter, the wire having at least one curved segment that extends away from the target magnet when the wire is advanced distally through the lumen in the target magnet. In another example the wire is constructed with an inner core coupled to a distal end of one or more extensible arms, and an outer coil coupled to a proximal end of one or more arms, such that by moving the inner core proximally relative to the outer coil, the arms bend outward, and by moving the inner core distally relative to the outer coil, the arms straighten so that they can pass through the lumen in the target magnet.

In another example, the target catheter has a primary shaft and an accessory lumen extending at least partially through the target magnet.

In a preferred example, the patient is placed on its dorsum on the operating table. Both the femoral artery and femoral vein are cut down and access to the vasculature is accomplished. A transesophageal echo (TEE) probe is placed in the esophagus and measurements of the mitral annulus such as the minor axis, major axis, circumference and areas in the surgical view are performed prior to percutaneous intervention. The femoral artery is accessed with an introducer (for example, a 14F×13 cm Cook Performer Introducer) while the femoral vein is accessed with a larger introducer (for example, an 18F×13 cm Cook performer introducer.) A 0.035″ diameter guidewire is position across the aortic valve with fluoroscopic guidance. A pigtail is then delivered into the ventricle. The C arm of the fluoroscope is oriented such that the long-axis view of the left side of the heart such that both the chambers of atrium and ventricle are not obstructed by aortic outflow. Upon removal of the guidewire, a cardiac ventriculogram is performed by injecting a 50% dye contrast into the left ventricle while cineradiography (CINE) is being acquired. The review of the CINE acquisition is played in slow motion and one or more frames are chosen that show the mitral annulus from the left ventricle side in the long axis view, whereby the mitral leaflet is at the point of closing. These frames can be saved on adjacent screens of the live fluoroscope video screen as reference. The 0.035″ guidewire is advanced beyond the distal tip of the pigtail and the pigtail is removed. A ventricular catheter with a target magnet attached to its distal end is introduced retrograde into the femoral artery and allowed to cross the aortic valve with the aid of the guidewire in its lumen. The catheter is then deflected so as to place the distal target magnet between the posterior leaflet such as P2 and the ventricular wall. The magnet distal surface is further position inferiorly of and in close contact adjacent to the mitral posterior annulus with the aid of fluoroscopy and TEE guidance. An outer steerable guiding sheath (for example, a 13.8F Oscor Destino Twist steerable guiding sheath) with an angle tip dilator is advanced from the femoral vein into the superior vena cava with a 0.035″ guidewire. The guidewire is removed and a transseptal access device such as a Brockenbrough needle is inserted into the dilator. The needle is then advanced to the tip of the dilator towards the fossa ovalis under fluoroscopy and TEE guidance. The needle tip is advanced to puncture the septum and the guidewire is advanced beyond the septum. The access device is withdrawn, and the tip of the dilator followed by the guiding sheath is advanced through the septum with the aid of the guidewire. The dilator and guidewire are removed from the sheath. If necessary, a syringe filled with 50% dye contrast can be used to inject dye into the sheath and out the tip of the catheter to ensure that the distal end of the sheath is in the left atrium. With TEE observing the mitral annulus from the surgical view, the tip of the 13.8F sheath is defected towards the desired posterior annular location such as P2. The handle of the sheath is then connected to the coupling slide on the stability base and locked. The angle of tilt of the control rail on the stability based can be adjusted to match the access point of entry into the patient. An atrial catheter with a locating helical tissue anchor coupled to a torque tube is inserted into the lumen of a steerable sheath (for example, a 7F Oscor Destino Twist steerable guiding sheath.) The atrial catheter and sheath combination are advanced until the locating magnet exits the distal end of the outer steerable sheath. If necessary, the outer steerable sheath may be readjusted towards the desired posterior annular location if the atrial catheter introduction changed its location. The atrial catheter is then advanced and steered in the vicinity of the target magnet until the surfaces of both the locating magnet and target magnet attract due to their opposite polarities and are magnetically coupled. The atrial catheter can then be manipulated such that the locating magnet acts as a pivot or hinge with respect to the target magnet to allow the user to reorient a delivery direction or path of the atrial catheter relative to the stationary ventricular catheter so as to provide an optimal angle of approach for the helical anchor to be implanted towards and adjacent to the annulus under fluoroscopic view. The helical anchor is then advanced, and the torque tube is rotated to secure the anchor into the annular tissue. After the helical anchor is in the tissue, the atrial catheter and guiding sheath are withdrawn from the outer sheath leaving the torque tube coupled to the anchor. A guidewire with an angle tip is then advanced through the lumen of the ventricular catheter. When the curve tip exits that tip of the magnet, the axial orientation of the guidewire tip naturally orient itself along the annulus due to the tip being constantly pushed by the opening of the adjacent posterior leaflet. The delivery catheter with the template temporarily attached to a set of jaws is then advanced through the outer steerable sheath to enter the atrium with the torque tube as a guide. The torque tube enters from the center opening of the template and exits a rapid exchange side port of the delivery catheter. A double clamping torquer device is attached to the end of the torque tube such that the expandable element is within the device. The torquers are tightened to secure the device to the torque tube. Using appropriate C arm angle such as RAO 30 to see the annulus superiorly, the template is advanced towards the annulus with the arms are deflected proximally. The axial line of the template is rotated to match the annular line provided by the guidewire tip in the vent tube is pulled into the template to attach in place and secured by tabs on the proximal end of the helical anchor. As a result, the template pulls in annular tissue into its apex. With slight adjustment of the axial line of the template to the axial line of the guidewire, the arms are undeflected distally and placed on the annulus on either side of the helical anchor. The side anchors coupled with torque tubes are then turned into the tissue, securing the side anchors into the annulus. The template is then released from the jaws of the delivery catheter. The side anchors are released by pulling their respective lock wires. With the lock boss on the double clamping torquer device released, the proximal handle is turned in the indicated direction of the device until the expandable element is stretched, causing the lock wire the move proximally and decoupling the torque tube from the helical anchor. The delivery catheter and the torque tube are removed from the outer catheter. TEE measurements are made to determine movement of the annulus. Measurements show that the minor axis moved towards the anterior by 40% and the area of the annulus changed by 20%. The 13.8F catheter is removed from the femoral vein and the ventricular catheter is removed from the femoral artery. The introducer sheaths are then removed, and hemostasis is achieved. Patient is allowed to recuperate from the procedure.

What is claimed is:

1. A catheter system for delivering an anchor to a valve annulus in a heart valve, said catheter system comprising:
a locating catheter having a distal end configured to be advanced beneath the valve annulus near a target site on an upper surface of the valve annulus;
an anchor delivery catheter having a distal end configured to be advanced over the valve annulus to deliver an anchor along a delivery path to the target site, wherein the distal end of the locating catheter is configured with at least one magnetic element and the distal end of the anchor delivery catheter is configured with at least one magnetic element, wherein the at least one magnetic element on the locating catheter attracts the at least one magnetic element on the anchor delivery catheter to pivotally couple the distal end of the anchor delivery catheter to the distal end of the locating catheter, wherein the deliver catheter can be pivoted relative to the locating catheter to orient the delivery path away from the locating catheter.

2. The catheter system of claim 1, wherein the locating catheter is configured to be placed into a ventricle and the anchor delivery catheter is configured to be placed in the atrium.

3. The catheter system of claim 2, wherein the locating catheter is configured to be placed into a left ventricle beneath a mitral valve annulus and the anchor delivery catheter is configured to be placed in the left atrium above the mitral valve annulus.

4. The catheter system of claim 1, wherein each of the magnetic elements comprises a magnet.

5. The catheter system of claim 4, wherein one of the magnetic elements comprises a magnet and the other of the magnetic elements comprises a magnetizable structure.

6. The catheter system of claim 4, wherein each magnet is axially polarized with a distal tip of the magnet on the delivery catheter having a polarity opposite to a polarity of the distal tip of the magnet on the locating catheter.

7. The catheter system of claim 4, wherein one of the magnets is axially polarized and the other magnet is radially polarized.

8. The catheter system of claim 4, wherein at least one of the magnets comprises a shell magnet.

9. The catheter system of claim 8, wherein the shell magnet comprises a delivery lumen on the anchor delivery catheter.

10. The catheter system of claim 9, wherein the anchor delivery of the shell magnet is aligned with a delivery lumen on the anchor delivery catheter.

11. The catheter system of claim 4, wherein the magnet on the locating catheter comprises a blunt tip magnet.

12. The catheter system of claim 1, wherein the locating catheter is configured to fit between a valve leaflet and a ventricular wall when the valve is open, allowing the valve function without causing significant stenosis of the heart valve.

13. The catheter system of claim 1, wherein the locating catheter comprises a visual alignment marker at its distal tip, wherein said visual alignment marker is configured to allow a user to align the distal end of the locating catheter with the target site while the locating catheter is being advanced under visualization.

14. The catheter system of claim 13, wherein the visual alignment marker comprises an optical, a fluoroscopic, or an echogenic marker.

15. The catheter system of claim 13, wherein the visual alignment marker comprises a plurality of laterally extending arms.

16. The catheter system of claim 15, wherein the laterally extending arms are straight.

17. The catheter system of claim 15, wherein the laterally extending arms are curved.

18. The catheter system of claim 17, wherein the curved arms are on an axially extendable shaft.

19. The catheter system of claim 1, wherein the anchor comprises a helical anchor detachably secured to a rotatable drive shaft located in a lumen of the anchor delivery catheter.

20. A method for delivering an anchor to a heart valve annulus, said method comprising:
advancing a distal end of a locating catheter into a heart ventricle to a location beneath the valve annulus near a target site on of the valve annulus;
advancing a distal end of an anchor delivery catheter to a location over the upper surface of the valve annulus adjacent the target site, wherein the anchor delivery catheter is configured to deliver an anchor along a delivery path;
magnetically coupling the distal end of the anchor delivery catheter to the distal end of the locating catheter across the mitral valve annulus to selectively along the delivery path through the heart valve annus toward the locating catheter; and
advancing the anchor along the delivery path to the target site.

21. The method of claim 20, further comprising manipulating the anchor delivery catheter to align the delivery path with the target site on the valve annulus, wherein the magnetic coupling allows the distal end of the anchor delivery catheter to pivot relative to the distal end of the locating catheter while manipulating the anchor delivery catheter.

22. The method of claim 21, wherein the anchor delivery catheter is delivered through an introducer sheath into the left atrium.

23. The method of claim 22, wherein manipulating comprises axially advancing and retracting the anchor delivery catheter through the introducer sheath to pivot the distal end of the anchor delivery catheter and change the direction of the delivery path.

24. The method of claim 22, manipulating comprises advancing and retracting a distal end of the introducer sheath within the atrium to pivot the distal end of the anchor delivery catheter and change the direction of the delivery path.

25. The method of claim 20, wherein (1) the distal end of the locating catheter is aligned along a wall of the ventricle and (2) the distal end of the anchor delivery catheter and the delivery path are directed laterally outwardly relative to the locating catheter so that the delivery path intersects the annulus at a target site located radially outwardly of the catheter.

26. The method of claim 20, wherein the heart valve annulus comprises a mitral valve annulus.

27. The method of claim 20, wherein the heart valve annulus comprises an aortic valve annulus, a pulmonary valve annulus, or a tricuspid valve annulus.

28. The method of claim 20, wherein the locating catheter comprises flexible extensions extending radially from the catheter body approximate the distal tip of the catheter.

29. The method of claim 28, further comprising visualizing the flexible extensions to assist in positioning the locating catheter beneath the valve leaflet.

30. The method of claim 29, wherein the flexible extensions are visualized using fluoroscopy.

31. The method of claim 29, wherein the flexible extensions are visualized using ultrasonography.

32. The method of claim 29, wherein visualizing the extensions on the locating catheter is used to align an implant coupled to the atrial tissue anchor.
